(19) United States
(12) Patent Application Publication
(10) Pub. No.: US 2016/0038583 A1
(43) Pub. Date: Feb. 11, 2016

(54) INFLUENZA HEMAGGLUTININ VARIANTS AND USES THEREFOR

(71) Applicant: MedImmune, LLC, Gaithersburg, MD (US)

(72) Inventors: Hong Jin, Gaithersburg, MD (US); Zhongying Chen, Gaithersburg, MD (US)

(21) Appl. No.: 14/503,763

(22) Filed: Oct. 1, 2014

Related U.S. Application Data
(60) Provisional application No. 61/886,440, filed on Oct. 3, 2013.

(51) Int. Cl. A61K 39/145; C07K 14/005; C12N 7/00

(52) U.S. Cl. CPC A61K 39/145; C12N 7/00; C07K 14/005; C12N 2760/16121; C12N 2760/16122; C12N 2760/16123; C12N 2760/16134; A61K 2039/53

(57) ABSTRACT

The present invention features polynucleotides encoding hemagglutinin (HA) polypeptide variants of a wild-type A/Anhui/1/2013 HA polypeptide, H7N9 influenza A viruses comprising such modified HA polynucleotides, methods of growing such viruses, and immunogenic compositions comprising such polynucleotides.

### CROSS REFERENCE TO RELATED APPLICATIONS

This application claims benefit under 35 U.S.C. §119(e) of U.S. Provisional Application No. 61/886,440 filed Oct. 3, 2013. The above listed application is incorporated by reference herein in its entirety for all purposes.

### REFERENCE TO THE SEQUENCE LISTING

This application incorporates by reference a Sequence Listing submitted with this application as text file entitled FLU-118US1_SL created on Aug. 7, 2014 and having a size of 25.9 kilobytes.

### BACKGROUND OF THE INVENTION

Sporadic human infections with avian influenza A viruses, which usually occur after recent exposure to poultry, have caused a wide spectrum of illness, ranging from conjunctivitis and upper respiratory tract disease to pneumonia and multi-organ failure. A novel avian-origin reassortant influenza A virus (H7N9) was recently identified in China. Of 134 people infected with this virus, most experienced severe respiratory illness, and 43 of those infected died. Given that H7N9 can infect mammals, cause severe illness, and may become transmissible between humans, H7N9 poses a potential serious threat to human health. In fact, should these viruses become capable of spreading from human to human, they could spark a pandemic. In recent tests of human immunity to H7N9, only low levels of population immunity were observed. Thus, immunogenic compositions that could prevent or treat H7N9 viral infection are urgently required.

### SUMMARY OF THE INVENTION

As described below, the present invention features polynucleotides encoding hemagglutinin (HA) variants. The invention further includes H7N9 influenza A viruses comprising the modified HA polynucleotides of the invention, methods of growing such viruses, and immunogenic compositions comprising such viruses. In one aspect, the invention generally provides polynucleotides encoding modified HA polypeptides containing one or more mutations at an amino acid position selected from the group consisting of 123, 125, 149, 151, 184, 189, 190, 211, and 215 of an amino acid sequence of a wild-type A/Anhui/1/2013 virus HA polypeptide (set forth in SEQ ID NO: 1). A virus comprising a modified HA polypeptide will have enhanced growth in eggs relative to a reference virus (e.g., relative to the growth in eggs of a wild-type A/Anhui/1/2013 virus).

In another aspect, the invention provides an HA polynucleotide encoding SEQ ID NO: 3. In one embodiment, the HA polynucleotide contains or consists essentially of the nucleotide sequence set forth in SEQ ID NO: 4.

In a related aspect, the invention provides a modified HA polypeptide derived from a wild-type A/Anhui/1/2013 virus, the HA polypeptide containing one or more mutations at an amino acid position selected from the group consisting of 123, 125, 149, 151, 184, 189, 190, 211, and 215 of a wild-type A/Anhui/1/2013 virus HA amino acid sequence (SEQ ID NO: 1). A virus comprising a modified HA polypeptide will have enhanced growth in eggs relative to a reference virus (e.g., relative to the growth in eggs of a wild-type A/Anhui/1/2013 virus).

In another related aspect, the invention provides a modified HA polypeptide derived from a wild-type A/Anhui/1/2013 virus, the HA polypeptide containing at least one mutation at an amino acid position selected from 123, 125, 149, or 189 of the wild-type A/Anhui/1/2013 viral HA polypeptide sequence (SEQ ID NO: 1). A virus comprising a modified HA polypeptide will have enhanced growth in eggs relative to a reference virus (e.g., relative to the growth in eggs of a wild-type A/Anhui/1/2013 virus).

In another aspect, the invention provides a vector (e.g., pAD 3000) containing the HA polynucleotide of a previous aspect or any other aspect of the invention delineated herein. In one embodiment, the vector is an expression vector. In another embodiment, the expression vector comprises a promoter suitable for driving expression of the polynucleotide in a cell. In another aspect, the invention provides a cell containing the vector of a previous aspect or any other aspect of the invention delineated herein.

In another aspect, the invention provides a reassortant recombinant virus comprising a polynucleotide that encodes the modified HA polypeptide of a previous aspect or any other aspect of the invention delineated herein. In one embodiment, the reassortant recombinant virus comprises a polynucleotide encoding a neuraminidase polypeptide (e.g., a neuraminidase of wild-type A/Anhui/1/2013 virus). In another embodiment, the reassortant recombinant virus comprises six internal gene segments of a Master Donor Virus (MDV). In another embodiment, the MDV is cold adapted (ca) influenza A master donor virus (MDV-A).

In another aspect, the invention provides an isolated virus-like particle containing a modified HA polypeptide of a previous aspect, or any other aspect of the invention delineated herein.

In yet another aspect, the invention provides an immunogenic composition containing a polynucleotide encoding the modified HA polypeptide of a previous aspect or any other aspect of the invention delineated herein. In one embodiment, the composition further comprises a polynucleotide encoding an Influenza A neuraminidase polypeptide.

In still another aspect, the invention provides an immunogenic composition containing the reassortant recombinant virus of a previous aspect or the virus-like particle of a previous aspect.

In still another aspect, the invention provides an immunogenic composition containing a 6:2 reassortant virus containing a polynucleotide encoding a modified HA polypeptide of a previous aspect, a NA polypeptide of an Influenza A virus, and six internal proteins encoded by a MDV. In one embodiment, the MDV is cold adapted (ca) Influenza A virus (MDV-A). In an embodiment, the composition is formulated for intranasal or subcutaneous delivery.

In another aspect, the invention provides a method for inducing an immune response against an Influenza A virus, in particular an H7N9 virus, the method involving administering to a subject the immunogenic composition of any previous aspect or any other aspect of the invention delineated herein.

In yet another aspect, the invention provides a method of preventing or treating an Influenza A virus infection, the method involving administering to a subject having or at risk of acquiring an Influenza viral infection an effective amount of the immunogenic composition of any previous aspect or any other aspect of the invention delineated herein. In one embodiment, the invention provides a method of preventing or treating an H7N9 virus infection.

In another aspect, the invention provides a kit comprising the immunogenic composition of any previous aspect or any other aspect of the invention delineated herein. In one embodiment, the kit comprises instructions for using the composition to induce an immune response in a subject. In another embodiment, the kit comprises instructions for using the composition to prevent or treat an H7N9 viral infection.

In another aspect, the invention provides a method for increasing viral titer during H7N9 viral replication in eggs, the method involving replicating the virus of a previous aspect in an embryonated egg, where the viral titer is increased relative to the growth of a wild-type H7N9 virus in the egg. In one embodiment, growth of wild-type virus is undetectable. In another embodiment, growth of the virus is increased at least about 10-fold, 100-fold, or more relative to the growth of wild-type virus.

In various embodiments of the previous aspects, or any other aspect of the invention delineated herein, the modified HA polypeptide contains mutations at an amino acid position that is any one or more of 123, 125, 149, 151, 184, 189, 190, 211, and 215 of a wild-type A/Anhui/1/2013 virus amino acid sequence (SEQ ID NO: 1). In other embodiments, a modified HA polypeptide contains one or more of the following mutations: N123D, A125T, N149D, A151T, K184N, G189E, N190D, R211S, and N215D. In one embodiment of any previous aspect or any other aspect of the invention delineated herein, the modified HA polypeptide comprises N123D, A125T, N149D, and/or G189E mutations. In one embodiment of any previous aspect or any other aspect of the invention delineated herein, the modified HA polypeptide comprises N123D and/or G189E mutations. In one embodiment, the modified HA polypeptide of a previous aspect or any other aspect of the invention delineated herein further comprises an additional mutation at an amino acid position selected from the group consisting of 125, 149, 151, 184, 190, 211, and 215, where the mutation further enhances growth in eggs relative to a reference. In one embodiment, the reference is the growth in eggs of a wild-type A/Anhui/1/2013 virus. In another embodiment, the additional mutation is a N149D and/or A125T mutation. In one embodiment, the amino acid sequence of the modified HA polypeptide comprises or consists essentially of SEQ ID NO: 3. In various embodiments of the above aspects, the invention provides a polynucleotide that encodes a modified HA polypeptide of any previous aspect or of any other aspect of the invention delineated herein. In other embodiments of the above aspects, an immunogenic composition of an above aspect or any other aspect of the invention delineated herein is administered to a subject. In other embodiments of the above aspects or any other aspect of the invention, a virus of the invention comprises an MDV that is cold adapted (ca) Influenza A virus (MDV-A). In other embodiments of the above aspects, an immunogenic composition is formulated for intranasal or subcutaneous delivery.

The invention provides immunogenic compositions for inducing an immune response against an H7N9 influenza A virus in a subject and methods of using such compositions to prevent or treat an H7N9 viral infection in a subject (e.g., human) in need thereof. In particular embodiments, the invention provides a 6:2 reassortant virus comprising polynucleotides encoding hemagglutinin and neuraminidase polypeptides from H7N9 and 6 internal gene plasmids from MDV-A. The polynucleotide encoding the hemagglutinin polypeptide comprises egg adaptation sequence changes relative to the sequence of the wild-type (wt) virus hemagglutinin from a human isolate. Compositions and articles defined by the invention were isolated or otherwise manufactured in connection with the examples provided below. Other features and advantages of the invention will be apparent from the detailed description, and from the claims.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “modified hemagglutinin (HA) polypeptide” is meant a recombinant HA polypeptide, or fragment thereof, having at least one altered amino acid relative to a reference or wild type HA polypeptide. The sequence of an exemplary wild-type H7 HA polypeptide, which could serve as a reference sequence, is provided at SEQ ID NO: 1. The sequence of an exemplary modified H7 HA polypeptide is provided at SEQ ID NO: 3.

By “modified hemagglutinin (HA) polynucleotide” is meant a nucleic acid molecule that encodes a modified HA polypeptide or fragment thereof. The sequence of an exemplary modified H7 HA polynucleotide is provided at SEQ ID NO: 4. The sequence of a wild-type H7 HA polynucleotide is provided at SEQ ID NO: 2.

By “vRNA” is meant the viral RNA obtained from a virus such as the H7N9 virus described herein.

By “wild-type” or “wt” is meant the typical form of an organism, polypeptide, or polynucleotide as it occurs in nature. In one embodiment, a wild-type H7N9 virus is the A/Anhui/1/2013 clinical isolate.

By “mutation” is meant a permanent alteration in the sequence of a gene. Exemplary mutations include frameshift mutations, insertions, missense mutations, nonsense mutations, point mutations, silent mutations, duplications, deletions, or any other form of genetic alteration known in the art. Mutations may be introduced by recombinant methods, mutagenesis, by selecting for genetic alterations that have a desirable characteristic (e.g., enhancing growth of a virus in eggs), or by any other method known in the art.

By “effective amount of” is meant an amount of an immunogenic composition sufficient to induce or enhance an immune response in a subject. Levels of induced immunity can be monitored, e.g., by measuring amounts of neutralizing secretory and/or serum antibodies, e.g., by plaque neutralization, complement fixation, enzyme-linked immunosorbent assay, microneutralization assay or any other method known in the art. The effective amount of active compound(s) used to practice the present invention for prophylaxis or for therapeutic treatment of a disease varies depends upon the manner of administration, the age, body weight, and general health of the subject. Ultimately, the attending physician or veterinarian will decide the appropriate amount and dosage regimen. Such amount is referred to as an “effective” amount.

By “enhances growth in eggs” is meant any positive alteration in a growth characteristic of a modified H7N9 virus in eggs. For example, a mutation that enhances growth in eggs provides an increase in viral titer. In one embodiment, the viral titer is increased to a level of >8.0 log₁₀FFU/ml. In other embodiments, the mutation increases the production of vaccine by at least about a 5%, 10%, 15%, 20%, 25%, 30% or greater.

A “protective immune response” against influenza virus refers to an immune response exhibited by a subject (e.g., a human) that is protective against disease when the individual is subsequently exposed to and/or infected with wild-type influenza virus. In some instances, the wild-type (e.g., naturally circulating) influenza virus can still cause infection, but it cannot cause a serious or life-threatening infection. Typically, the protective immune response results in detectable levels of host engendered serum and secretory antibodies that are capable of neutralizing virus of the same strain and/or subgroup (and possibly also of a different, non-vaccine strain and/or subgroup) in vitro and in vivo.

By “ameliorate” is meant decrease, suppress, attenuate, diminish, arrest, or stabilize the development or progression of a disease. An exemplary disease is an H7N9 viral infection, and associated symptoms.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “disease” is meant any condition or disorder that damages or interferes with the normal function of a cell, tissue, or organ. Examples of diseases include an H7N9 viral infection and associated symptoms. In one embodiment, a disease is influenza or symptoms associated with an H7N9 viral infection.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “reference” is meant a standard or control condition.

A “reference sequence” is a defined sequence used as a basis for sequence comparison.

By “subject” is meant a mammal, including, but not limited to, a human or non-human mammal, such as a bovine, equine, canine, ovine, or feline.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

### BRIEF DESCRIPTION OF THE SEQUENCE LISTING

SEQ ID NO: 1 depicts the amino acid sequence of a wild-type HA polypeptide from the A/Anhui/1/2013 strain of the H7N9 virus. X123=N or D; X125=A or T; X149=N or D.

SEQ ID NO: 2 depicts the nucleic acid sequence of the polynucleotide encoding the wild-type HA polypeptide from A/Anhui/1/2013 strain of the H7N9 virus.

SEQ ID NO: 3 depicts the amino acid sequence of the HA polypeptide from the V7 variant of the A/Anhui/1/2013 strain of the H7N9 virus.

SEQ ID NO: 4 depicts the nucleic acid sequence of the polynucleotide encoding the modified HA polypeptide from the V7 variant of the A/Anhui/1/2013 strain of the H7N9 virus.

SEQ ID NO: 5 depicts the amino acid sequence of the wild-type NA polypeptide from the A/Anhui/1/2013 strain of the H7N9 virus.

SEQ ID NO: 6 depicts the nucleic acid sequence of the polynucleotide encoding the wild-type NA polypeptide from A/Anhui/1/2013 strain of the H7N9 virus.

SEQ ID NO: 7 depicts the amino acid sequence of the NA polypeptide from the V7 variant of the A/Anhui/1/2013 strain of the H7N9 virus.

SEQ ID NO: 8 depicts the nucleic acid sequence of the polynucleotide encoding the NA polypeptide from the V7 variant of the A/Anhui/1/2013 strain of the H7N9 virus.

### BRIEF DESCRIPTION OF THE DRAWINGS

FIG. 1A through FIG. 1F show plaque morphology of ca A/Anhui/1/2013 strain variants V1 to V6.

FIG. 2A through FIG. 2E show the plaque morphology of ca A/Anhui/1/2013 variants V7-V11.

FIG. 3A and FIG. 3B depicts an alignment of the nucleotide sequence encoding the HA polypeptide from wild type A/Anhui/1/2013 strain (set forth in SEQ ID NO: 2) with the nucleotide sequence encoding the HA polypeptide from the V7 variant of the A/Anhui/1/2013 strain (set forth in SEQ ID NO: 4). FIG. 3A—depicts nucleotides 1 to 900; FIG. 3B—depicts nucleotides 901 to 1733.

FIG. 4 depicts an alignment of the amino acid sequence from the HA polypeptide from wild type A/Anhui/1/2013 strain (set forth in SEQ ID NO: 1) with the amino acid sequence of the HA polypeptide from the V7 variant of the A/Anhui/1/2013 strain (set forth in SEQ ID NO: 3).

FIG. 5A and FIG. 5B depict an alignment of the nucleotide sequence encoding the NA polypeptide from wt A/Anhui/1/2013 strain (set forth in SEQ ID NO: 6) with the nucleotide sequence encoding the NA polypeptide from the V7 variant of the A/Anhui/1/2013 strain (set forth in SEQ ID NO: 8. FIG. 5A—depicts nucleotides 1 to 840; FIG. 5B—depicts nucleotides 841 to 1444.

FIG. 6 depicts an alignment of the amino acid sequence of the NA polypeptide from wild type A/Anhui/1/2013 strain (set forth in SEQ ID NO: 5) with the amino acid sequence of the NA polypeptide from the V7 variant of the A/Anhui/1/2013 strain (set forth in SEQ ID NO: 7).

FIG. 7 is an image of viral polypeptides separated on a polyacrylamide gel and stained with Coomassie blue. Lane 1: 6:2 PR8-A/Anhui/1/2013(V1); Lane 2: 6:2 PR8-A/Anhui/1/2013(V7); Lane 3: A/shanghai/2/2013 (RG32A); Lane 4: 6:2 MDVA-A/Anhui/1/2013(V1); Lane 5: 6:2 MDVA-A/Anhui/1/2013(V7).

### DETAILED DESCRIPTION OF THE INVENTION

As described below, the present invention features polynucleotides encoding hemagglutinin (HA) variants. The invention further includes H7N9 influenza A viruses comprising the modified HA polynucleotides of the invention, methods of growing such viruses, and immunogenic compositions comprising such viruses. In one aspect, the invention generally provides polynucleotides encoding modified HA polypeptides containing one or more mutations at an amino acid position selected from the group consisting of 123, 125, 149, 151, 184, 189, 190, 211, and 215 of an amino acid sequence of an HA polypeptide of a wild-type A/Anhui/1/2013 virus (set forth in SEQ ID NO: 1). A virus comprising a modified HA polypeptide will have enhanced growth in eggs relative to a reference virus (e.g., relative to the growth in eggs of a wild-type A/Anhui/1/2013 virus). The immunogenic compositions of the invention are capable of inducing an immune response against an H7N9 influenza A virus. The invention comprises methods of using such compositions to generate a prophylactic or therapeutic immune response in a subject.

The present invention is based, at least in part, on the discovery of modified HA polypeptides in 6:2 vaccine strains derived from H7N9 A/Anhui/2013 isolates that replicate to high titers in chicken eggs. The modified HA polynucleotides encode HA polypeptides having at least one mutation at any one or more of amino acid positions N123D, G189E, R211S, A151T, K184N, N190D, and N215D, where the numbering corresponds to the numbering of a reference HA polypeptide of the H7N9 A/Anhui/2013 isolate. In one embodiment, the modified HA polynucleotide encodes an HA polypeptide comprising at least one or all of D at position 123, A at position 125, N at position 149, and E at position 189. A virus comprising a modified polynucleotide encoding the modified polypeptide of the invention grows well in eggs, has the correct antigenicity and is immunogenic in ferrets.

### Influenza a (H7N9) Virus

Influenza viruses are enveloped RNA viruses that belong to the family of Orthomyxoviridae. There are three types of influenza viruses: A, B and C. Human influenza A and B viruses cause seasonal epidemics of disease in the United States. Type A influenza infects other species as well, including birds, pigs, and other animals. In addition to annual epidemics, influenza viruses are the cause of infrequent pandemics. For example, influenza A viruses caused pandemics in 1918, 1957 and 1968. Due to the lack of pre-formed immunity against the major viral antigen, hemagglutinin (HA), pandemic influenza viruses can affect greater than 50% of the population in a single year and often cause more severe disease than seasonal influenza viruses. A stark example is the pandemic of 1918, in which an estimated 50-100 million people died from influenza.

In early 2013, a novel Avian-origin influenza A (H7N9) virus infection was identified in Shanghai, China. This H7N9 virus had not previously been detected in humans or animals. Human infection with H7N9 virus was associated with severe respiratory illness and death. This outbreak was the first time that avian influenza subtype (H7N9) was identified in human. By May of 2013, China reported the identification of human H7N9 cases in eight provinces, including the province of Anhui.

### Influenza Viruses

Influenza viruses are typically made up of an internal ribonucleoprotein core containing a segmented single-stranded RNA genome and an outer lipoprotein envelope lined by a matrix protein. The genome of influenza viruses is composed of eight segments of linear (−) strand ribonucleic acid (RNA), encoding immunogenic hemagglutinin (HA) and neuraminidase (NA) proteins, and six internal core polypeptides: a nucleocapsid nucleoprotein (NP); matrix proteins (M); non-structural proteins (NS); and 3 RNA polymerase (PA, PB1, PB2) proteins. During replication, genomic viral RNA is transcribed into (+) strand messenger RNA and (−) strand genomic cRNA in the nucleus of the host cell. Each of the eight genomic segments is packaged into ribonucleoprotein complexes that contain, in addition to the RNA, a nucleocapsid nucleoprotein (NP) and a polymerase complex (PB1, PB2, and PA). The hemagglutinin molecule includes a surface glycoprotein and can bind to N-AcetylNeuraminic acid (NeuNAc), also known as sialic acid, on host cell surface receptors. Hemagglutinin is made up of two subunits, HA1 and HA2 and the entire structure is about 550 amino acids in length and has a molecular weight of about 61 kD. Neuraminidase molecules cleave terminal sialic acid residues from cell surface receptors of the influenza virus, thereby releasing virions from infected cells. Neuraminidase also removes sialic acid from newly made hemagglutinin and neuraminidase molecules.

Influenza is typically grouped into influenza A and influenza B categories, and sometimes a less common C category. Influenza A viruses are negative-sense, single-stranded, segmented RNA viruses. Influenza A viruses are divided into subtypes on the basis of their hemagglutinin (H1 to H17) and neuraminidase (N1 to N10) activity. Influenza variants may also be characterized as low or highly pathogenic based on their ability to cause disease in poultry. Only two influenza A virus subtypes (i.e., H1N1, and H3N2) are currently in general circulation among humans.

Influenza A and influenza B viruses each contain eight segments of single stranded RNA with negative polarity. The influenza A genome encodes eleven polypeptides. Segments 1-3 encode three polypeptides, making up an RNA-dependent RNA polymerase. Segment 1 encodes the polymerase complex protein PB2. The remaining polymerase proteins PB1 and PA are encoded by segment 2 and segment 3, respectively. In addition, segment 1 of some influenza strains encodes a small protein, PB1-F2, produced from an alternative reading frame within the PB1 coding region. Segment 4 encodes the hemagglutinin (HA) surface glycoprotein involved in cell attachment and entry during infection. Segment 5 encodes the nucleocapsid nucleoprotein (NP) polypeptide, the major structural component associated with viral RNA. Segment 6 encodes a neuraminidase (NA) envelope glycoprotein. Segment 7 encodes two matrix proteins, designated M1 and M2, which are translated from differentially spliced mRNAs. Segment 8 encodes NS1 and NS2, two nonstructural proteins, which are translated from alternatively spliced mRNA variants.

The eight genome segments of influenza B encode 11 proteins. The three largest genes code for components of the RNA polymerase, PB1, PB2 and PA. Segment 4 encodes the HA protein. Segment 5 encodes NP. Segment 6 encodes the NA protein and the NB protein. Both proteins, NB and NA, are translated from overlapping reading frames of a bicistronic mRNA. Segment 7 of influenza B also encodes two proteins: M1 and BM2. The smallest segment encodes two products: NS1 is translated from the full length RNA, while NS2 is translated from a spliced mRNA variant.

Different strains of influenza can be categorized based upon, for example, the ability of influenza to agglutinate red blood cells (RBCs or erythrocytes). Antibodies specific for particular influenza strains can bind to the virus and, thus, prevent such agglutination. Assays determining strain types based on such inhibition are typically known as hemagglutinin inhibition assays (HI assays or HAI assays) and are standard and well known methods in the art to characterize influenza strains. As used herein, “HI assay” and “HAI assay” are used interchangeably to refer to such assays.

The influenza virus particle envelope protein hemagglutinin (HA) binds not only to sialic acid receptors on cells, but also to erythrocytes (red blood cells). This property is called hemagglutination, and is the basis of a rapid assay to determine levels of influenza virus present in a sample. To conduct the assay, two-fold serial dilutions of a virus are prepared, mixed with a specific amount of red blood cells, and added to the wells of a plastic tray. The red blood cells that are not bound by influenza virus sink to the bottom of a well. The red blood cells that are attached to virus particles form a lattice that coats the well. The assay provides a quick indicator of the relative quantities of virus particles in a sample.

The assay can be easily modified to determine the level of antibodies to influenza virus present in serum samples (HAI assay). Fixed amount of virus are added to each well of a 96-well plate, (equivalent to 32-64 HA units). Two-fold dilutions of serum to be tested are added to each dilution series along a row of wells. Finally, red blood cells are added and incubated for 30 minutes. The basis of the HAI assay is that antibodies to influenza virus will prevent attachment of the virus to red blood cells. Therefore hemagglutination is inhibited when antibodies are present. The highest dilution of serum that prevents hemagglutination is called the HAI titer of the serum. If the serum contains no antibodies that react with the virus, then hemagglutination will be observed in all wells. Likewise, if antibodies to the virus are present, hemagglutination will not be observed until the antibodies are sufficiently diluted. By determining HI titers and comparing them with influenza attack rates in populations, it is possible to calculate the significance of the HI antibody titer with respect to susceptibility to influenza virus infection.

In typical HAI assays, sera to be used for typing or categorization, which is often produced in ferrets, is added to erythrocyte samples in various dilutions. Optical determination is then made by determining whether the erythrocytes are clumped together (i.e., agglutinated) or are suspended (i.e., non-agglutinated). If the cells are not clumped, then agglutination did not occur due to the inhibition from antibodies in the sera that are specific for that influenza. Thus, the types of influenza are defined as being within the same strain. In some cases, one strain is described as being “like” the other strain. For example, if two samples are within four-fold titer of one another as measured by an HAI assay, then they can be described as belonging to the same strain (e.g., both belonging to the “New Caledonia” strain, or both being “Moscow-like” strains). In other words, strains are typically categorized based upon their immunologic or antigenic profile. An HAI titer is typically defined as the highest dilution of a serum that completely inhibits hemagglutination. See, e.g., Schild, et al., (1973) Bull. Wld. Hlth. Org. 48:269-278.

As used herein, the term “similar strain” should be taken to indicate that a first influenza virus is of the same or related strain as a second influenza virus. In typical embodiments such relation is commonly determined through use of an HAI assay. Influenza viruses that fall within a four-fold titer of one another in an HAI assay are, thus, of a “similar strain.” Other assays are known in the art for the determination of similar strains, e.g., FRID, neutralization assays, and the like. The polypeptides provided herein (and the nucleic acids that encode the polypeptides provided herein) also comprise such similar strains in the various plasmids, vectors, viruses, methods, and the like herein. Thus, unless the context clearly dictates otherwise, descriptions herein of particular sequences (e.g., those in the sequence listing) or fragments thereof also should be considered to include sequences from similar strains to those (i.e., similar strains to those strains having the sequences in those plasmids, vectors, viruses, and the like herein). Also, it will be appreciated that the NA and HA polypeptides within such similar strains are, thus, “similar polypeptides” when compared between “similar strains.”

From the above it will be appreciated that the modified polynucleotides encoding the modified polypeptides provided herein (and nucleic acids encoding the polypeptides provided herein) not only include polynucleotides comprising the specific sequences listed herein, but also such polynucleotides within various vectors (e.g., those used for plasmid reassortment and rescue, described in further detail below), as well as hemagglutinin and neuraminidase sequences within the same strains as the sequences listed herein. Also, such same strains that are within various vectors (e.g., typically ones used for plasmid reassortment and rescue such as A/Ann Arbor/6/60 or B/Ann Arbor/1/66, A/Puerto Rico/8/34, B/Leningrad/14/17/55, B/14/5/1, B/USSR/60/69, B/Leningrad/179/86, B/Leningrad/14/55, or B/England/2608/76, and the like) are also included.

### Influenza Vaccine Production

Most influenza virus vaccines used in the United States and Europe are grown in embryonated eggs. After harvest from the eggs, the preparations are formaldehyde-inactivated, purified and chemically disrupted with a nonionic detergent. This preparation is feasible for only (high-yielding) influenza A viruses. Even with influenza A viruses, the 6:2 reassortants (HA and NA from recently circulating strains and the remaining 6 genes from A/PR/8/34 virus) are sometimes difficult to obtain. Once the process of reassortment is completed, the strain is then passaged in embryonated eggs to allow for egg adaptation and growth enhancement.

Current methods of live vaccine production provide for a cold-adapted, temperature-sensitive, and highly attenuated master strain. This master strain is then updated by reassortment with viruses more closely related to the currently circulating influenza strains. The resulting vaccine strains (both A and B types) are 6:2 reassortants with the 6 nonsurface protein genes derived from the cold-adapted master strains and the HA and NA from circulating A and B viruses, reflecting the changing antigenicity. These cold-adapted influenza virus vaccines are easily administered by nasal spray.

Many influenza vaccines are produced using reverse genetics, infectious influenza viruses can be obtained using plasmid DNAs transfected into tissue culture cells. This technology permits the construction of high-yield 6:2 seed viruses by mixing the 6 plasmid DNAs from a good-growing laboratory strain with the HA and NA DNAs obtained by cloning relevant genes from currently circulating viruses. Thus, within about a 1- to 2-week period, the appropriate seed viruses could be generated for distribution to manufacturers. The backbones of the 6:2 recombinant viruses could be prepared, tested, and distributed in advance.

Reassortant viruses can be referred to herein as a chimeric viruses or recombinant viruses. The term “chimeric” or “chimera,” when referring to a virus, indicates that the virus includes genetic and/or polypeptide components derived from more than one parental viral strain or source. Similarly, the term “chimeric” or “chimera,” when referring to a viral protein, indicates that the protein includes polypeptide components (i.e., amino acid subsequences) derived from more than one parental viral strain or source. As will be apparent herein, such chimeric viruses are typically reassortant/recombinant viruses. Thus, in some embodiments, a chimera can include, for example, a sequence (e.g., of HA and/or NA) from an H7N9 virus placed into a backbone comprised of, or constructed/derived from a Master Donor Virus.

The term “recombinant” indicates that the material (e.g., a nucleic acid or protein) has been artificially or synthetically (non-naturally) altered by human intervention. The alteration can be performed on the material within, or removed from, its natural environment or state. Specifically, e.g., an influenza virus is recombinant when it is produced by the expression of a recombinant nucleic acid. For example, a “recombinant nucleic acid” is one that is made by recombining nucleic acids, e.g., during cloning, DNA shuffling or other procedures, or by chemical or other mutagenesis; a “recombinant polypeptide” or “recombinant protein” is a polypeptide or protein which is produced by expression of a recombinant nucleic acid; and a “recombinant virus,” e.g., a recombinant H7N9 influenza virus, is produced using at least one recombinant nucleic acid.

The term “reassortant,” when referring to a virus (typically herein, an H7N9 influenza virus), indicates that the virus includes genetic and/or polypeptide components derived from more than one parental viral strain or source. For example, a 7:1 reassortant includes 7 viral genome segments (or gene segments) derived from a first parental virus, and a single complementary viral genome segment, e.g., encoding a hemagglutinin or neuraminidase described herein. A 6:2 reassortant includes 6 genome segments, most commonly the 6 internal genome segments from a first parental virus, and two complementary segments, e.g., hemagglutinin and neuraminidase genome segments, from one or more different parental viruses (e.g., H7N9 influenza virus, such as a modified HA and NA derived from A/Anhui/1/2013). As mentioned above, reassortant viruses also can, depending upon context herein, be termed as “chimeric” and/or “recombinant.”

In some cases, recombinant and reassortant vaccines are produced in cell culture using a vector system (see, e.g., U.S. Pat. No. 8,012,736 and U.S. Pat. No. 8,114,415). Such systems can be useful for rapid production vaccines corresponding to one or many selected antigenic strains of virus, e.g., either A or B strains, various subtypes or substrains, and the like, e.g., comprising the modified HA and NA polynucleotides disclosed herein (e.g., H7N9 influenza virus, such as a modified HA polynucleotide and/or NA polynucleotide derived from A/Anhui/1/2013). Typically, cultures are maintained in a system, such as a cell culture incubator, under controlled humidity and 00₂, at constant temperature using a temperature regulator, such as a thermostat to insure that the temperature does not exceed 35° C. Reassortant influenza viruses can be readily obtained by introducing a subset of vectors corresponding to genomic segments of a master influenza virus, in combination with complementary segments derived from strains of interest (e.g., HA and NA antigenic variants herein). Typically, master strains are selected on the basis of desirable properties relevant to vaccine administration. For example, for vaccine production, e.g., for production of a live attenuated vaccine, the master donor virus strain may be selected for an attenuated phenotype, cold adaptation and/or temperature sensitivity. As explained elsewhere herein and, e.g., in U.S. Pat. No. 8,012,736, various embodiments herein utilize A/Ann Arbor (AA)/6/60 or B/Ann Arbor/1/66 or A/Puerto Rico/8/34, or B/Leningrad/14/17/55, B/14/5/1, B/USSR/60/69, B/Leningrad/179/86, B/Leningrad/14/55, or B/England/2608/76 influenza strain as a “backbone” upon which to add HA and/or NA genes (e.g., (e.g., a modified HA polynucleotide and/or NA polynucleotide derived from A/Anhui/1/2013, or other polynucleotides listed herein and variants thereof) to create desired reassortant viruses. Thus, for example, in a 6:2 reassortant, 2 genes (i.e., NA and HA) would be from the influenza strain(s) against which an immunogenic reaction is desired (e.g., H7N9 influenza virus, such as a modified HA and/or NA derived from A/Anhui/1/2013), while the other 6 genes would be from the Ann Arbor strain, or other backbone strain. The Ann Arbor virus is useful for its cold adapted, attenuated, temperature sensitive attributes. Additionally, the HA and NA polynucleotides and variants thereof provided herein are capable of reassortment with a number of other virus genes or virus types (e.g., a number of different “backbones” such as A/Puerto Rico/8/34, for example, containing other influenza genes present in a reassortant, namely, the non-HA and non-NA genes). In some embodiments, the reassortants can be 7:1 reassortants. In such cases, either the HA or the NA is from a different strain than the backbone or MDV strain.

In some embodiments, viruses are temperature sensitive, cold adapted and/or attenuated. The terms “temperature sensitive”, “cold adapted” and “attenuated” as applied to viruses (typically used as vaccines or for vaccine production) are known in the art. For example, the term “temperature sensitive” (ts) indicates, for example, that a virus exhibits a 100 fold or greater reduction in titer at 39° C. relative to 33° C. for influenza A strains, or that the virus exhibits a 100 fold or greater reduction in titer at 37° C. relative to 33° C. for influenza B strains. The term “cold adapted” (ca) indicates that the virus exhibits growth at 25° C. within 100 fold of its growth at 33° C., while the term “attenuated” (att) indicates that the virus replicates in the upper airways of ferrets, but is not detectable in their lung tissues, and does not cause influenza-like illness in the animal. It will be understood that viruses with intermediate phenotypes, i.e., viruses exhibiting titer reductions less than 100 fold at 39° C. (for A strain viruses) or 37° C. (for B strain viruses), or exhibiting growth at 25° C. that is morethan 100 fold than its growth at 33° C. (e.g., within 200 fold, 500 fold, 1000 fold, 10,000 fold less), and/or exhibit reduced growth in the lungs relative to growth in the upper airways of ferrets (i.e., partially attenuated) and/or reduced influenza like illness in the animal, are also useful viruses and can be used in conjunction with the HA and NA sequences herein.

Thus, methods described herein can utilize growth, e.g., in appropriate culture conditions, of virus strains (both A strain and B strain influenza viruses) with desirable properties relative to vaccine production (e.g., attenuated pathogenicity or phenotype, cold adaptation, temperature sensitivity, and the like) in vitro in cultured cells. Influenza viruses can be produced by introducing a plurality of vectors incorporating cloned viral genome segments into host cells, and culturing the cells at a temperature not exceeding 35° C., for example. When vectors including an influenza virus genome are transfected, recombinant viruses suitable as vaccines can be recovered by standard purification procedures.

The term “introduced” when referring to a heterologous or isolated nucleic acid refers to the incorporation of a nucleic acid into a eukaryotic or prokaryotic cell where the nucleic acid can be incorporated into the genome of the cell (e.g., chromosome, plasmid, plastid or mitochondrial DNA), converted into an autonomous replicon, or transiently expressed (e.g., transfected mRNA). The term includes such methods as “infection,” “transfection,” “transformation,” and “transduction.” A variety of methods can be employed to introduce nucleic acids into cells, including electroporation, calcium phosphate precipitation, lipid mediated transfection (lipofection), and the like.

The term “host cell” means a cell that contains a heterologous nucleic acid, such as a vector or a virus, and supports the replication and/or expression of the nucleic acid. Host cells can be prokaryotic cells, such as E. coli, or eukaryotic cells, such as yeast, insect, amphibian, avian or mammalian cells, including human cells. Non-limiting examples of host cells include Vero (African green monkey kidney) cells, BHK (baby hamster kidney) cells, primary chick kidney (PCK) cells, Madin-Darby Canine Kidney (MDCK) cells, Madin-Darby Bovine Kidney (MDBK) cells, 293 cells (e.g., 293T cells), and COS cells (e.g., COS1, COS7 cells), and the like. In certain embodiments, host cells can include eggs (e.g., hen eggs, embryonated hen eggs, and the like). In some cases, 9 to 12-day old embryonated hen eggs are used with the methods herein.

Using the vector system and methods herein, reassortant viruses incorporating six internal gene segments of a strain selected for its desirable properties with respect to vaccine production, and the immunogenic HA and NA segments from a selected, e.g., pathogenic strain such as those provided herein (e.g., H7N9 influenza virus, such as a modified HA and NA derived from A/Anhui/1/2013), can be rapidly and efficiently produced in tissue culture and/or eggs. Thus, the system and methods described herein are useful for the rapid production in cell culture and/or eggs of recombinant and reassortant H7N9 viral strains, including viruses suitable for use as vaccines, including live attenuated vaccines, such as, for example, vaccines suitable for intranasal administration.

In such embodiments, typically, a single Master Donor Virus (MDV) strain is selected for the H7N9 viral strain. In certain cases where a live attenuated vaccine is produced, the Master Donor Virus strain is typically chosen for its favorable properties, e.g., temperature sensitivity, cold adaptation and/or attenuation, relative to vaccine production. For example, Master Donor Strains include such temperature sensitive, attenuated and cold adapted strains of A/Ann Arbor/6/60 and B/Ann Arbor/1/66, respectively, as well as others mentioned herein or known in the art.

In some cases, a selected master donor type A virus (MDV-A), or master donor type B virus (MDV-B), can be produced from a plurality of cloned viral cDNAs constituting the viral genome. Embodiments include those where recombinant viruses are produced from eight cloned viral cDNAs. Eight viral cDNAs representing the selected MDV-A or MDV-B sequences of PB2, PB1, PA, NP, HA, NA, M and NS can be cloned into a bi-directional expression vector. The term “vector”, as used herein, refers to the means by which a nucleic acid can be propagated and/or transferred between organisms, cells, or cellular components. Vectors include plasmids, viruses, bacteriophages, pro-viruses, phagemids, transposons, artificial chromosomes, and the like, that replicate autonomously or can integrate into a chromosome of a host cell. A vector also can be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that is not autonomously replicating. In some embodiments, the vectors herein are plasmids. An “expression vector” is a vector, such as a plasmid, that is capable of promoting expression, as well as replication of a nucleic acid incorporated therein. Typically, the nucleic acid to be expressed is “operably linked” to a promoter and/or enhancer, and is subject to transcription regulatory control by the promoter and/or enhancer. As used herein, “expression of a gene” or “expression of a nucleic acid” typically means transcription of DNA into RNA (optionally including modification of the RNA, e.g., splicing) or transcription of DNA into mRNA, translation of RNA into a polypeptide (possibly including subsequent modification of the polypeptide, e.g., post-translational modification), or both transcription and translation, as indicated by the context. A “bi-directional expression vector” is characterized by two alternative promoters oriented in the opposite direction relative to a nucleic acid situated between the two promoters, such that expression can be initiated in both orientations resulting in, for example, transcription of both plus (+) or sense strand, and negative (−) or antisense strand RNAs. Bi-directional expression vectors may be used with the methods provided herein, such as for example pAD3000, such that the viral genomic RNA can be transcribed from an RNA polymerase I (pol I) promoter from one strand and the viral mRNAs can be synthesized from an RNA polymerase II (pol II) promoter from the other strand. Optionally, any gene segment can be modified, including the HA segment, for example, to enhance growth in eggs.

Following transfection of plasmids bearing the eight viral cDNAs into appropriate host cells, e.g., Vero cells, co-cultured MDCK/293T or MDCK/COS7 cells, infectious recombinant MDV-A virus comprising a modified HA of A/Anhui/1/2013 can be recovered. Using the plasmids and methods described herein and, e.g., in U.S. Pat. No. 8,012,736; U.S. Pat. No. 8,114,415; Hoffmann, E. (2000) Proc. Natl. Acad. Sci. USA, 97(11):6108-6113; U.S. Pat. No. 6,951,754; and U.S. Pat. No. 6,544,785, the polypeptides provided herein are useful for generating 6:2 reassortant influenza vaccines by co-transfection of the 6 internal genes (PB1, PB2, PA, NP, M and NS) of a selected donor virus (e.g., MDV-A, MDV-B) together with the HA and NA polypeptides derived from different corresponding type influenza viruses (e.g., H7N9 influenza virus, such as a modified HA and/or NA polypeptides derived from A/Anhui/1/2013). For example, the HA polynucleotide segment can be selected from an H7N9 influenza virus, such as A/Anhui/1/2013. Similarly, the HA segment can be selected from a strain with emerging relevance as a pathogenic strain such as those described herein. Reassortants incorporating seven genome segments of the MDV and either the HA or NA gene of a selected strain (7:1 reassortants) can also be produced. It will be appreciated, and as is detailed throughout, molecules provided herein can optionally be combined in any desired combination. For example, the HA and/or NA sequences herein can be placed, for example, into a reassortant backbone such as A/AA/6/60, B/AA/1/66, A/Puerto Rico/8/34 (i.e., PR8), and the like, in 6:2 reassortants or 7:1 reassortants, for example. Thus, as explained in more detail below, there can be 6 internal genome segments from the donor virus and 2 genome segments from a second strain, such as, for example a wild-type or modified strain that is different from the donor strain. Such 2 genome segments are typically the HA and NA genes. For 7:1 reassortants, in which there are 7 genome segments from the donor virus and 1 genome segment (either HA or NA) from a different viral strain, such as, for example a wild-type or modified strain that is different from the donor strain. Often, for 6:2 or 7:1 reassortants, the HA and/or NA is derived from a strain to which an immune response is desired. Also, it will be appreciated that the polypeptide and/or nucleic acid sequences herein can be combined in a number of means in different embodiments herein. Thus, any of the sequences herein can be present singularly in a 7:1 reassortant (i.e., a sequence herein combined with 7 donor virus genome segments) and/or can be present with another sequence provided herein in a 6:2 reassortant. Within such 6:2 reassortants, any of the sequences provided herein can be present with any other sequence herein. Typically, 6:2 reassortants include HA and NA polypeptides from the same strain. For example, certain embodiments can include a 6:2 reassortant having 6 internal genome segments from a donor virus such as, for example A/AA/6/60, and HA and NA genome segments described herein. In some cases, such reassortant viruses include HA and NA genome segments from similar strains.

Polynucleotides encoding the modified HA polypeptides provided herein are optionally utilized in plasmid reassortant vaccines such as those described herein and ts, cs, ca, and/or att viruses and vaccines. The HA and NA sequences provided herein are not limited to specific vaccine compositions or production methods, and can, thus, be utilized in substantially any vaccine type or vaccine production method which utilizes strain specific HA and NA antigens.

### FluMist

One exemplary influenza vaccine is FluMist (MedImmune Vaccines Inc., Mt. View, Calif.), which is a live, attenuated vaccine that protects children and adults from influenza illness (Belshe et al. (1998) N Engl J Med 338:1405-1412; Nichol et al. (1999) JAMA 282:137-144). In certain embodiments, the methods and compositions provided herein can be adapted to/used with production of FluMist vaccine. However, the modified polynucleotides, methods and compositions described herein are also adaptable to production of similar or different viral vaccines.

FluMist vaccines contain, for example, HA polynucleotides (e.g., encoding a modified HA polypeptide of the invention) and NA polynucleotides derived from the wild-type strains to which the vaccine is addressed (or, in some instances, from related strains) along with six polynucleotides encoding PB1, PB2, PA, NP, M, and NS, from a common master donor virus (MDV). The polynucleotides encoding the modified HA and NA polynucleotides disclosed herein can thus be included in various FluMist formulations. The MDV for influenza A strains of FluMist (MDV-A), was created by serial passage of the wild-type A/Ann Arbor/6/60 (A/AA/6/60) strain in primary chicken kidney tissue culture at successively lower temperatures (Maassab (1967) Nature 213:612-614). MDV-A replicates efficiently at 25° C. (ca, cold adapted), but its growth is restricted at 38 and 39° C. (ts, temperature sensitive). Additionally, this virus does not replicate in the lungs of infected ferrets (att, attenuated). The ts phenotype is believed to contribute to the attenuation of the vaccine in humans by restricting its replication in all but the coolest regions of the respiratory tract. The stability of this property has been demonstrated in animal models and clinical studies. In contrast to the ts phenotype of influenza strains created by chemical mutagenesis, the ts property of MDV-A does not revert following passage through infected hamsters, or is shed in isolates from children (see Murphy & Coelingh (2002) Viral Immunol 15:295-323).

### Administration of Immunogenic Compositions

The modified polynucleotides, modified polypeptides, methods, and compositions provided herein can be used to generate in a subject an immune response against an H7N9 virus. In general, recombinant and reassortant viruses prepared with the modified polynucleotides described herein can be administered prophylactically in an immunologically effective amount to stimulate an immune response. Vaccines comprising the recombinant and reassortant viruses of the invention may optionally comprise an appropriate carrier or excipient.

Typically, the carrier or excipient for vaccines provided herein is a pharmaceutically acceptable carrier or excipient, such as sterile water, aqueous saline solution, aqueous buffered saline solutions, aqueous dextrose solutions, aqueous glycerol solutions, ethanol, allantoic fluid from uninfected hen eggs (i.e., normal allantoic fluid or NAF), or combinations thereof. The preparation of such solutions ensuring sterility, pH, isotonicity, and stability is effected according to protocols established in the art. Generally, a carrier or excipient is selected to minimize allergic and other undesirable effects, and to suit the particular route of administration, e.g., subcutaneous, intramuscular, intranasal, and the like.

Also provided herein are methods for stimulating the immune system of an individual to produce a protective immune response against influenza virus. In such methods, an immunologically effective amount of a recombinant influenza virus provided herein, an immunologically effective amount of a modified polypeptide provided herein, and/or an immunologically effective amount of a modified nucleic acid provided herein is administered to the individual and may, optionally be in a physiologically acceptable carrier.

Generally, the influenza viruses provided herein are administered in a quantity sufficient to stimulate an immune response specific for one or more strains of influenza virus (i.e., against the H7N9 strain). Typically, administration of the influenza virus elicits a protective immune response to such strains. Dosages and methods for eliciting a protective immune response against one or more influenza strains are known in the art. See, e.g., U.S. Pat. No. 5,922,326; Wright et al. (1982) Infect. Immun. 37:397-400; Kim et al. (1973) Pediatrics 52:56-63; and Wright et al. (1976) J. Pediatr. 88:931-936. For example, influenza viruses are provided in the range of about 1-1000 HID₅₀ (human infectious dose), i.e., about 10⁵-10⁸ pfu (plaque forming units) per dose administered. Typically, the dose will be adjusted within this range based on factors which include age, physical condition, body weight, sex, diet, time of administration, and other clinical factors, for example. The prophylactic vaccine formulation can be systemically administered, e.g., by subcutaneous or intramuscular injection using a needle and syringe, or a needle-less injection device. In some cases, the vaccine formulation is administered intranasally, either by drops, large particle aerosol (greater than about 10 microns), or spray into the upper respiratory tract. While any of the above routes of delivery results in a protective systemic immune response, intranasal administration confers the added benefit of eliciting mucosal immunity at the site of entry of the influenza virus. For intranasal administration, attenuated live virus vaccines are often used. The vaccines compris e.g., attenuated, cold adapted and/or temperature sensitive recombinant or reassortant influenza viruses. While stimulation of a protective immune response with a single dose is typical, additional dosages can be administered, by the same or different route, to achieve the desired prophylactic effect.

Typically, an attenuated recombinant influenza virus provided herein, as used in a vaccine, is sufficiently attenuated such that symptoms of infection, or at least symptoms of serious infection, will not occur in most individuals immunized (or otherwise infected) with the attenuated influenza virus. In some instances, the attenuated influenza virus can still produce symptoms of mild illness (e.g., mild upper respiratory illness) and/or of dissemination to unvaccinated individuals. However, its virulence is sufficiently abrogated such that severe lower respiratory tract infections typically do not occur in the vaccinated or incidental host.

In some cases, an immune response can be stimulated by ex vivo or in vivo targeting of dendritic cells with influenza viruses containing the sequences provided herein. For example, proliferating dendritic cells can be exposed to viruses in a sufficient amount and for a sufficient period of time to permit capture of the influenza antigens by dendritic cells. The cells are then transferred into a subject to be vaccinated by standard intravenous transplantation methods.

While stimulation of a protective immune response with a single dose is typical, additional dosages can be administered, by the same or different route, to achieve the desired prophylactic effect. In neonates and infants, for example, multiple administrations may be required to elicit sufficient levels of immunity. Administration can continue at intervals throughout childhood, as necessary to maintain sufficient levels of protection against wild-type influenza infection. Similarly, adults who are particularly susceptible to repeated or serious influenza infection, such as, for example, health care workers, day care workers, family members of young children, the elderly, and individuals with compromised cardiopulmonary function may require multiple immunizations to establish and/or maintain protective immune responses. Levels of induced immunity can be monitored, for example, by measuring amounts of neutralizing secretory and serum antibodies, and dosages adjusted or vaccinations repeated as necessary to elicit and maintain desired levels of protection.

Optionally, the formulation for prophylactic administration of the influenza viruses also contains one or more adjuvants for enhancing the immune response to the influenza antigens. Suitable adjuvants include: complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, bacille Calmette-Guerin (BCG), Corynebacterium parvum, and the synthetic adjuvants QS-21 and MF59.

In some cases, prophylactic vaccine administration of influenza viruses can be performed in conjunction with administration of one or more immunostimulatory molecules.

The above described methods can be useful for therapeutically and/or prophylactically treating a disease or disorder, typically influenza, including an H7N9 viral infection and/or symptoms thereof, by introducing a vector comprising a heterologous polynucleotide encoding a therapeutically or prophylactically effective HA and/or NA polypeptide (or peptide).

Although vaccination of an individual with an attenuated influenza virus of a particular strain of a particular subgroup can induce cross-protection against influenza viruses of different strains and/or subgroups, cross-protection can be enhanced, if desired, by vaccinating the individual with attenuated influenza virus from at least two (i.e. bivalent), at least three (i.e. trivalent), or at least four (i.e. tetravalent) influenza virus strains or substrains, e.g., at least two of which may represent a different subgroup. For example, vaccinating an individual with at least four strains or substrains of attenuated influenza virus (i.e. tetravalent vaccine).

Additionally, vaccine combinations can optionally include mixes of pandemic vaccines and non-pandemic strains. Vaccine mixtures (or multiple vaccinations) can include components from human strains and/or non-human influenza strains (e.g., avian and human). Similarly, the attenuated influenza virus vaccines provided herein can optionally be combined with vaccines that induce protective immune responses against other infectious agents. In some embodiments, a vaccine provided herein is a trivalent vaccine comprising three reassortant influenza viruses. In particular embodiments, an HA polypeptide can include any of the amino acid substitutions described herein, including, for example, a mutation selected from the group consisting of N123D, A125T, N149D, A151T, K184N, G189E, N190D, R211S, and N215D, where the modifications are at positions corresponding to amino acid positions in SED ID NO: 1. In particular embodiments, an HA polypeptide comprises a combination of mutations, including N123D, A125T, N149D, A151T, K184N, G189E, N190D, R211S, and N215D.

### Production of Recombinant Virus

Negative strand RNA viruses can be produced and recovered using a recombinant reverse genetics approach (U.S. Pat. No. 5,166,057). Such a method was originally applied to produce influenza viral genomes (Luytjes et al. (1989) Cell 59:1107-1113; Enami et al. (1990) Proc. Natl. Acad. Sci. USA 92:11563-11567), and has been successfully applied to a wide variety of segmented and nonsegmented negative strand RNA viruses, e.g., rabies (Schnell et al. (1994) EMBO J. 13: 4195-4203); VSV (Lawson et al. (1995) Proc. Natl. Acad. Sci. USA 92: 4477-4481); measles virus (Radecke et al. (1995) EMBO J. 14:5773-5784); rinderpest virus (Baron & Barrett (1997) J. Virol. 71: 1265-1271); human parainfluenza virus (Hoffman & Banerjee (1997) J. Virol. 71: 3272-3277; Dubin et al. (1997) Virology 235:323-332); SV5 (He et al. (1997) Virology 237:249-260); canine distemper virus (Gassen et al. (2000) J. Virol. 74:10737-44); and Sendai virus (Park et al. (1991) Proc. Natl. Acad. Sci. USA 88: 5537-5541; Kato et al. (1996) Genes to Cells 1:569-579). Recombinant influenza viruses produced according to such methods are contemplated herein, as are recombinant influenza virus comprising one or more nucleic acids and/or polypeptides provided herein. Influenza viruses in general (and those provided herein) are negative stranded RNA viruses. Thus, when influenza viruses are described herein as comprising one or more sequences provided herein, it is to be understood that the corresponding negative stranded RNA version of each sequence are referred to as well. The nucleotide sequences provided herein typically comprise DNA versions (e.g., coding plus sense) of the genes (along with some untranslated regions in the nucleotide sequences, in some cases). Conversions between RNA and DNA sequences can be performed, for example, by changing U to T or T to U. Other sequence conversions, e.g., from a nucleotide sequence to the corresponding amino acid sequence or to a corresponding complementary sequence (whether DNA or RNA), also can be performed using methods known in the art. Also, when such HA and/or NA sequences are described within DNA vectors, e.g., plasmids, the corresponding DNA version of the sequences are typically to be understood. Nucleic acids provided herein thus include the explicit sequences in the sequence listings herein, as well as the complements of such sequences (both RNA and DNA), the double stranded form of the sequences provided herein, the corresponding RNA forms of the sequences provided herein (either as the RNA complement to the explicit sequence provided herein or as the RNA version of the sequence provided herein, e.g., of the same sense, but comprised of RNA, with U in place of T.

### Isolation, Cloning, Mutagenesis and Expression of Biomolecules of Interest

Various types of cloning and mutagenesis methods can be used with the methods herein, e.g., to produce and/or isolate, e.g., novel or newly isolated HA and/or NA molecules and/or to further modify/mutate the polynucleotides encoding the HA and NA molecules provided herein. As used herein, the term “isolated” refers to a biological material, such as a virus, a nucleic acid or a protein, which is substantially free from components that normally accompany or interact with it in its naturally occurring environment. The isolated biological material optionally comprises additional material not found with the biological material in its natural environment, e.g., a cell or wild-type virus. For example, if the material is in its natural environment, such as a cell, the material can have been placed at a location in the cell (e.g., genome or genetic element) not native to such material found in that environment. For example, a naturally occurring nucleic acid (e.g., a coding sequence, a promoter, an enhancer, and the like) becomes isolated if it is introduced by non-naturally occurring means to a locus of the genome (e.g., a vector, such as a plasmid or virus vector, or amplicon) not native to that nucleic acid. Such nucleic acids are also referred to as “heterologous” nucleic acids. An isolated virus, for example, is in an environment (e.g., a cell culture system, or purified from cell culture) other than the native environment of wild-type virus (e.g., the nasopharynx of an infected individual).

In some embodiments, isolated nucleic acids, polypeptides and/or viruses can be further mutated. Mutagenesis methods include but are not limited to site-directed, random point mutagenesis, homologous recombination (DNA shuffling), mutagenesis using uracil containing templates, oligonucleotide-directed mutagenesis, phosphorothioate-modified DNA mutagenesis, mutagenesis using gapped duplex DNA and the like. Additional suitable mutagenesis methods include point mismatch repair, mutagenesis using repair-deficient host strains, restriction-selection and restriction-purification, deletion mutagenesis, mutagenesis by total gene synthesis, double-strand break repair, and the like. Mutagenesis, e.g., involving chimeric constructs, is also included in the methods herein. In some embodiments, mutagenesis can be guided by known information of the naturally occurring molecule or altered or mutated naturally occurring molecule, e.g., sequence, sequence comparisons, physical properties, crystal structure or the like.

Oligonucleotides for use in mutagenesis (e.g., mutating libraries of the HA and/or NA molecules provided herein, or altering such) are typically synthesized chemically according to the solid phase phosphoramidite triester method described by Beaucage and Caruthers ((1981) Tetrahedron Letts 22(20):1859-1862) using an automated synthesizer, as described in Needham-VanDevanter et al. ((1984) Nucleic Acids Res 12:6159-6168). In addition, essentially any nucleic acid can be custom or standard ordered from any of a variety of commercial sources. Similarly, peptides and antibodies can be custom ordered from any of a variety of sources.

Also provided herein are host cells and organisms comprising an HA and/or NA polynucleotide or polypeptide, or other polypeptide and/or nucleic acid provided herein or such HA and/or NA or other polynucleotides within various vectors such as 6:2 reassortant influenza viruses, plasmids in plasmid rescue systems, and the like. Host cells can be transformed, transduced or transfected with the vectors provided herein, which can be, for example, a cloning vector or an expression vector. The vector can be, for example, in the form of a plasmid, a bacterium, a virus, a naked polynucleotide, or a conjugated polynucleotide. The vectors can be introduced into cells and/or microorganisms by standard methods including electroporation (see, From et al. (1985) Proc Natl Acad Sci USA 82: 5824), infection by viral vectors, high velocity ballistic penetration by small particles with the nucleic acid either within the matrix of small beads or particles, or on the surface (Klein et al. (1987) Nature 327: 70-73).

Several well-known methods of introducing target nucleic acids into bacterial cells are available, any of which can be used with the methods herein. These include, for example, fusion of the recipient cells with bacterial protoplasts containing the DNA, electroporation, projectile bombardment, and infection with viral vectors, and the like. Bacterial cells can be used to amplify the number of plasmids containing DNA constructs. The bacteria are grown to log phase and the plasmids within the bacteria can be isolated by a variety of methods known in the art. In addition, a plethora of kits are commercially available for the purification of plasmids from bacteria, (see, e.g., EASYPREP™, FLEXIPREP™, both from Pharmacia Biotech; STRATACLEAN™, from Stratagene; and QIAPREP™ from Qiagen). The isolated and purified plasmids are then further manipulated to produce other plasmids, used to transfect cells or incorporated into related vectors to infect organisms. Typical vectors contain transcription and translation terminators, transcription and translation initiation sequences, and promoters useful for regulation of the expression of the particular target nucleic acid. The vectors optionally comprise generic expression cassettes containing at least one independent terminator sequence, sequences permitting replication of the cassette in eukaryotes, or prokaryotes, or both, (e.g., shuttle vectors) and selection markers for both prokaryotic and eukaryotic systems. Vectors are suitable for replication and integration in prokaryotes, eukaryotes, or both. (See, Giliman and Smith (1979) Gene 8:81; Roberts, et al., Nature (1987) 328:731; Schneider, B., et al. (1995) Protein Expr Purif 6435:10; Ausubel, Sambrook, Berger (all supra)). A catalogue of Bacteria and Bacteriophages useful for cloning is provided, e.g., by the American Type Culture Collection (ATCC; The ATCC Catalogue of Bacteria and Bacteriophage (1992) Gherna et al. (eds.). Additional basic procedures for sequencing, cloning and other aspects of molecular biology and underlying theoretical considerations are known in the art.

### Increasing Peak Titer in Eggs

In some embodiments, the HA modifications are substitutions that can increase peak titer in embryonated eggs for a reassortant or recombinant influenza virus having a modified HA polypeptide described herein. The peak titer can be increased between about 1.5-fold to about 40-fold, for example 2-fold, 4-fold, 8-fold, 10-fold, 20-fold or 30-fold relative to the same reassortant or recombinant influenza having an unmodified HA polypeptide.

In some cases, peak titer can be further increased for reassortants having a native or modified neuraminidase (NA) polypeptide from the same or a different viral strain as the HA polypeptide. In some cases, the HA polypeptide is an avian influenza polypeptide, such as H7N9. In some embodiments, the reassortant or recombinant viruses described herein include an HA or NA polypeptide from a viral strain that typically grows well in embryonated eggs.

The modified HA polypeptides described herein can include additional amino acid substitutions. In some embodiments, the additional amino acid substitutions are substitutions that can further increase peak titer in embryonated eggs for a reassortant or recombinant influenza virus having a modified HA polypeptide.

### Silent Variations

Due to the degeneracy of the genetic code, any of a variety of nucleic acid sequences encoding polypeptides and/or viruses provided herein are optionally produced, some which can bear lower levels of sequence identity to the HA and NA nucleic acid and polypeptide sequences herein. Many amino acids are encoded by more than one codon. For example, the codons AGA, AGG, CGA, CGC, CGG, and CGU all encode the amino acid arginine. Thus, at every position in a nucleic acid where an arginine is specified by a codon, the codon can be altered to any of the corresponding codons described above without altering the encoded polypeptide. It is understood that U in an RNA sequence corresponds to T in a DNA sequence. Such “silent variations” are one species of “conservatively modified variations,” discussed below. Each codon in a nucleic acid (except ATG, which is ordinarily the only codon for methionine, and TTG, which is ordinarily the only codon for tryptophan) can be modified by standard techniques to encode a functionally identical polypeptide. Accordingly, each silent variation of a nucleic acid which encodes a polypeptide is implicit in any described sequence. The sequences provided herein, therefore, explicitly provide each and every possible variation of a nucleic acid sequence encoding a polypeptide provided herein that could be made by selecting combinations based on possible codon choices. These combinations are made in accordance with the standard triplet genetic code as applied to the nucleic acid sequence encoding a hemagglutinin or a neuraminidase polypeptide herein. All such variations of every nucleic acid herein are specifically provided and described by consideration of the sequence in combination with the genetic code. Such silent substitutions can be made using the methods herein.

### Kits

The invention provides kits for the treatment or prevention of an H7N9 viral infection. In one embodiment, the kit includes a therapeutic or prophylactic composition containing an effective amount of an immunogenic composition (e.g., a reassortant influenza virus comprising a polynucleotide encoding a modified HA polypeptide) in unit dosage form. In some embodiments, the kit comprises a device (e.g., nebulizer, metered-dose inhaler) for immunogenic composition dispersal or a sterile container which contains a therapeutic or prophylactic immunogenic composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired, an immunogenic composition of the invention is provided together with instructions for administering the immunogenic composition to a subject having or at risk of developing an H7N9 viral infection. The instructions will generally include information about the use of the composition for the treatment or prevention of a H7N9 infection. In other embodiments, the instructions include at least one of the following: description of the therapeutic/prophylactic agent; dosage schedule and administration for treatment or prevention of H7N9 infection or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### Generation of 6:2 Reassortant Vaccine Variants from vRNA of A/Anhui/1/2013

Working with wild-type (wt) A/Anhui/1/2013 strain of H7N9 virus requires biosafety level 3 containment (BLS-3 containment). Thus, viral RNA isolated from egg-amplified wt A/Anhui/1/2013 strain of H7N9 was obtained from the Centers for Disease Control and Prevention (CDC) for cloning of the HA and NA genes. The HA and NA gene segments of wt A/Anhui/1/2013 were amplified using reverse transcription polymerase chain reaction (RT-PCR) using primers that are universal to the HA and NA gene end sequences and cloned into the plasmid vector pAD3000 (Hoffman (2000) Proc. Natl. Acad. Sci. USA 97:6108-6113). Site-directed mutagenesis was performed to introduce specific changes into the HA genes using the QuikChange® Site-Directed Mutagenesis kit (Agilent Technologies, Santa Clara, Calif.) and the HA sequence was confirmed by sequencing analyses.

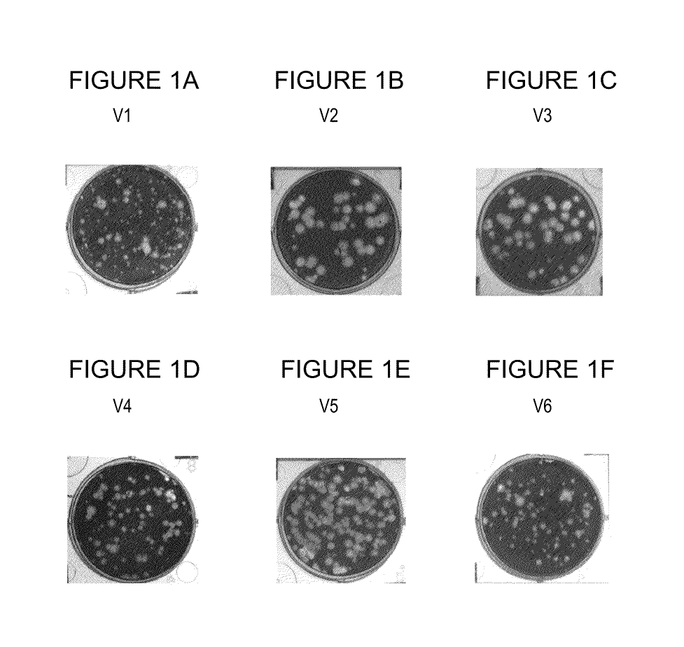

The 6:2 reassortant vaccine strains were generated by co-transfecting 8 cDNA plasmids encoding the HA and NA of the H7N9 virus and the 6 internal gene segments of cold adapted (ca) A/Ann Arbor/6/60 (MDV-A, master donor virus for type A influenza virus) into co-cultured 293T and MDCK cells. The vaccine strains used for manufacture are produced in serum-free Vero/CEK cells by electroporation. Viruses were propagated in the allantoic cavities of 10- to 11-day-old embryonated chicken eggs. To determine the peak titer of each virus in eggs, an additional viral amplification in eggs was performed and the viral titers were examined by the fluorescence focus assay (FFA). Virus titers were measured by the fluorescence focus assay using an anti-NP monoclonal antibody and expressed as log₁₀FFU (fluorescent focus units Unit)/ml (Forrest et al. (2008) Clin Vaccine Immunol 15:1042-1053). Virus plaque morphology was examined by plaque assay as previously described (Jin et al., (2003) Virology 306, 18-24). The HA and NA sequences of the rescued viruses were verified by sequencing of RT-PCR cDNAs amplified from vRNA. FIG. 1A through FIG. 1F shows the plaque morphology of ca A/Anhui/1/2013 strain variants V1 to V6.

The nucleotide sequence encoding the HA and NA polypeptides, and the amino acid sequence of the HA and NA polypeptide of wt H7N9 virus was downloaded from the Global Initiative on Sharing All Influenza Data (GISAID) database. The HA and NA gene segments were cloned from the viral RNA following RT-PCR. Alignment of the sequences obtained for the NA revealed that the NA clones had the same sequence as the wt virus. From 20 HA clones analyzed, 6 variants were isolated: V1 (5%) contained the same sequence as the original wt sequence, V2 (45%) had a N149D change, V3 (25%) had a N123D/N149D double mutation, V4 (5%) had a N123D change, V5 (15%) and V6 (5%) contained single mutations of A125T and N190D, respectively. Table 1, below, depicts the sequence changes in the HA polypeptides obtained from translating the vRNA and variants V1 to V6, and the peak titers obtained for variants V1 to V6.

TABLE 1
HA Sequence Changes and Virus Titers of ca A/Anhhui/1/2013
(H7N9) variants

| | Amino acid at the HA | | | | | |
|---|---|---|---|---|---|---|
| | position (H3#)^{a.} | | | | | |
| | 123 | 125 | 149 | 190 | | Peak titer in eggs |
| 6:2 variant | (133) | (135) | (158) | (199) | % Clones | (log₁₀ FFU/mL) |
| Wt | N | A | N | N | n/a | n/a |
| vRNA | N/D | A/T | D/N | | n/a | n/a |
| V1 | | | | | 5% | 7.3 |
| V2 | | | D | | 45% | 8.2 |
| V3 | D | | D | | 25% | 8.4 |
| V4 | D | | | | 5% | 7.7 |
| V5 | | T | | | 15% | 8.1 |
| V6 | | | D | 5% | 7.9 | D |

^{a.}Amino acid changes from the wt virus are shown.

As shown in Table 1, above, the rescued variants had different levels of replication in eggs with a titer ranging from 7.3 to 8.4 log₁₀FFU/ml. The 6:2 reassortant A/Anhui/1/2013 (V1) vaccine virus with the original HA sequence grew poorly in eggs and formed tiny plaques in MDCK cells (FIG. 1A). Egg adaptation HA sequence changes are required for virus to grow efficiently in eggs. V2 and V3 had the highest titers in eggs, indicating that the N149D change greatly improved the vaccine virus growth in eggs (FIG. 1B and FIG. 10). The V5 variant (A125T) that introduced a potential glycosylation site at N123 also improved the virus titer to a level of >8.0 log₁₀FFU/ml (FIG. 1E). The V4 variant (FIG. 1D) had a lower titer and the HA protein gene contained an additional mutation of G189E after a second egg passage, thus this variant was not selected for further evaluation in ferrets. The mutation was introduced into V4 to make an additional variant V7 as described below.

Plasmids representing these different HA sequences were combined with the NA plasmid and the 6 internal protein gene plasmids from MDV-A and transfected into 293/MDCK cells. The transfection supernatants containing the 6:2 reassortant viruses were inoculated into chicken embryonated eggs for virus propagation.

### Example 2

### Evaluation of Vaccine Variants for their Immunogenicity and Antigenicity in Ferrets

Male and female ferrets (8-12 weeks old; Simonson, Gilroy, Calif.) in groups of 3 were used to assess virus replication in the respiratory tracts and for vaccine immunogenicity. Ferrets were housed individually and inoculated intranasally with 7.0 log₁₀FFU of virus per 0.2 ml dose. Three days after infection, ferrets were euthanized, and the lungs and nasal turbinates (NT) were harvested. Virus titers in the lung and NT were determined by the EID₅₀ assay and expressed as 50% egg infectious dose per gram of tissue (log₁₀EID₅₀/g). Ferrets that were assigned for immunogenicity studies were bled on days 14, 21 and 28 days postinfection and sera were assessed for antibody titers by the hemagglutination inhibition (HAI) assay. Ferret antiserum against wt A/Anhui/1/2013 and the BPL-inactivated A/Anhui/1/2013 for antigenicity reference were obtained from CDC.

H7N9-specific antibody level in post-infected ferret sera against homologous and heterologous viruses was determined by HAI assay. Prior to the assay, ferret sera were treated with receptor-destroying enzyme (RDE) (Denka Seiken, Tokyo, Japan) that was reconstituted in 10 mL of 0.9% NaCl per vial. 0.1 mL serum was mixed with 0.15 mL RDE and incubated at 37° C. for 18 hr and adjusted to a final 1:4 dilution by adding 0.15 mL of 0.9% sodium citrate followed by incubation at 56° C. for 45 min. 25 μl of serial diluted RDE-treated serum samples were mixed with 4 HA units of tested viruses (25 μl). After 30 min of incubation, 50 μl of 0.5% chicken red blood cells (cRBC) was added and incubated for 45 minutes to determine the HAI titer. The HAI titers are presented as the reciprocal value of the highest serum dilution that inhibited hemagglutination.

The nucleic acid substitutions in the polynucleotide encoding HA result in amino acid substitutions in the head region of the HA trimer structure, some of which may alter the viral immunogenic and antigenic properties. To examine the immunogenicity and antigenicity of the variants, ferrets were inoculated with 7.0 log₁₀FFU of V1, V2, V3, V5 or V6 intranasally in 0.2 ml of dose volume. The post-infection serum samples were collected on day 14 and antibody titers were evaluated by hemagglutination inhibition (HAI) assay, the results of which are shown in Table 2, below.

TABLE 2
Immunogenicity and Antigenicity of ca A/Anhui/1/2013 HA Variants

| | | GMT HAI titer of ferret serum against | | | | | |
|---|---|---|---|---|---|---|---|
| | | A/Anhui/1/2013 | | | | | |
| Virus | HA sequence | Wt¹ | V1 | V2 | V3 | V5 | V6 |
| wt¹ | wt | 256 | 128 | n/a | n/a | n/a | n/a |
| (BPL) | | | | | | | |
| V1 | wt | 256 | 81 | 16 | 10 | 16 | 32 |
| V2 | N149D | 256 | 203 | 128 | 40 | 27 | 102 |
| V3 | N123/N149D | n/a | n/a | 64 | 40 | 23 | n/a |
| V5 | A125T | 128 | 40 | 13 | 16 | 19 | 23 |
| V6 | N190D | 256 | 128 | n/a | n/a | 23 | 64 |

n/a: not done
¹Ferret antiserum against wt A/Anhui/1/2013 and the BPL-inactivated A/Anhui/1/2013 were obtained from CDC.

The V1, V2 and V6 variants all elicited good antibody titers (GMT HAI titers 64-128) against the homologous virus. The V3 and V5 induced lower geometric mean titer (GMT) HAI antibody titers of 40 and 19, respectively. The antiserum against wt A/Anhui/1/2013 (obtained from CDC) cross-reacted well to V1. Similarly, the antiserum against V1 cross-reacted well to the β-propiolactone (BPL)-inactivated wt A/Anhui/1/2013 (obtained from CDC), confirming that V1 containing the wt HA sequence is antigenically identical to the wt virus and can be used as a reference virus for antigenicity test. Anti-V1 ferret serum cross-reacted well to each variant. V2 and V3 cross-reacted to V1 with a titer of 4-8-fold lower than the homologous titer, indicating that the N149D change affected viral antigenicity. The H7 149 residue corresponds to H3#158 and H1N1pdm#155. The G155E change in H1N1pdm has been shown to alter the viral antigenicity (Chen et al. (2010) J. Virol. 84(1): 44-51). The ferret antisera against V5 and V6 cross-reacted well to V1, indicating that the A125T and N190D changes did not significantly change viral antigenicity.

### Example 3

### Improvement of the Growth of Vaccine Variants in Eggs

None of the six variants had the ideal characteristics for vaccine manufacture. The variants with titers >8.0 log₁₀FFU/ml had altered antigenicity (V2 and V3) or low immunogenicity (V5). Thus, further improvement was needed to generate vaccine variants that grew well in eggs and induced good immunogenicity without altering antigenicity.

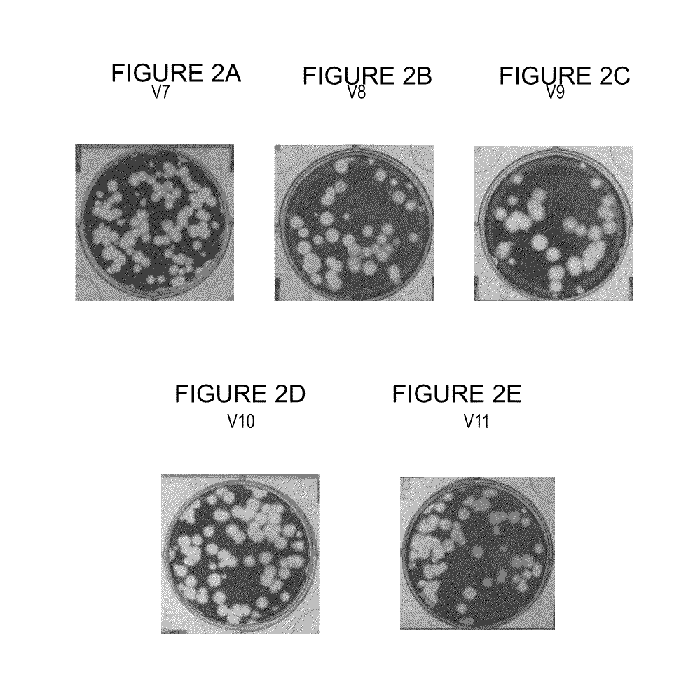

As seen in FIG. 1A to FIG. 1F, V1, V4 and V6 formed tiny or small plaques in MDCK cells. However, after further egg passages, the viruses formed much larger plaques, indicating that additional egg adaptation sequence changes improved virus growth and produced the larger plaques. The virus from the larger plaques was isolated and expanded in eggs to confirm the higher growth in eggs. The polynucleotide encoding HA of each of the large plaque morphology isolates was sequenced. Translation of the nucleotide sequences indicated that the HA polypeptide of the isolates that produced large plaque morphology have single amino acid changes at one of the following positions: G189E from V4, N215D from V6, and A151T, R211S or K184N from V1. Each of the identified mutations was introduced into the HA of V4, V6 or V1 and additional vaccine variants (V7-V11) were rescued. FIG. 2A through FIG. 2E show the plaque morphology of ca A/Anhui/1/2013 strain variants V7 to V11. The rescued viruses were examined for growth in eggs.

TABLE 3
ca A/Anhui/1/2013 HA variants identified from egg/MDCK adaptation
and introduced by reverse genetics

| | | | | | | | | Peak titer in |
|---|---|---|---|---|---|---|---|---|
| | Amino acid at HA position (H3#) | | | | | | | eggs |
| 6:2 | 123 | 151 | 184 | 189 | 190 | 211 | 215 | (log₁₀ FFU/ |
| variants | (133) | (160) | (193) | (198) | (199) | (220) | (224) | mL) |
| wt | N | A | K | G | N | R | N | |
| V7 | D | | | E | | | | 8.7 |
| V8 | | | | | D | | D | 8.2 |
| V9 | | T* | | | | | | 8.3 |
| V10 | | | | | | S | | 8.2 |
| V11 | | | N | | | | | 8.1 |

*A151T change introduces a potential N-glycosylation site at N149 (H3#158)

As shown in Table 3, above, all the variants exhibited higher titer (>8.0 log 10 FFU/ml) than the parental viruses, among which V7 (N123D/G189E) showed the highest titer of 8.7 log₁₀FFU/ml. Therefore, the V7 variant was selected and further tested in ferrets to evaluate its qualification as a vaccine candidate.

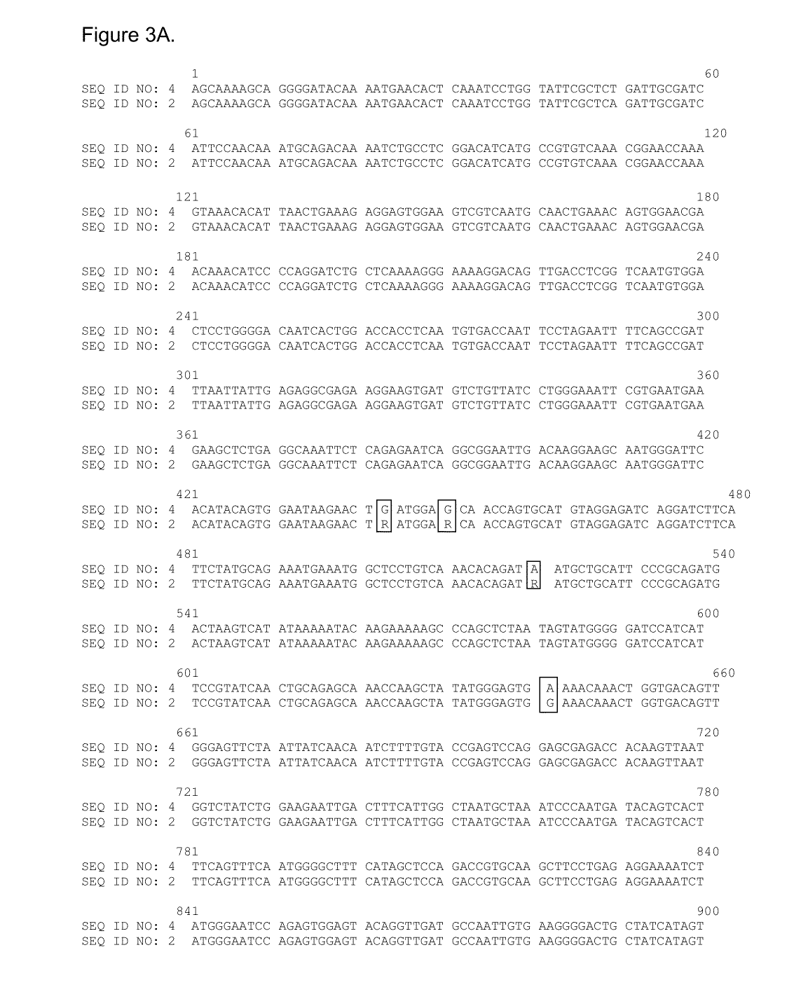

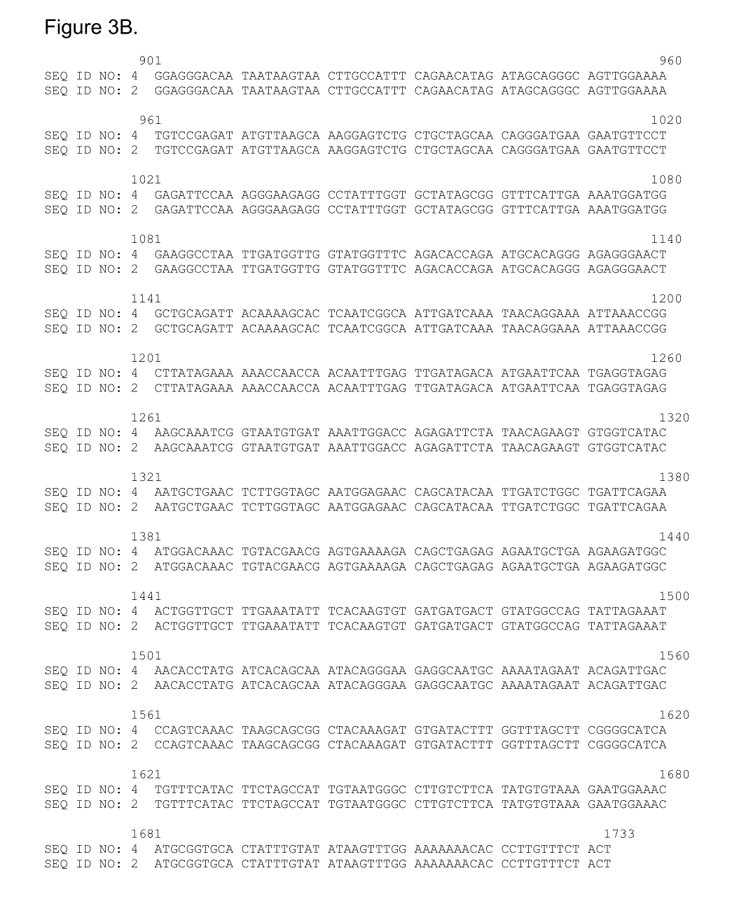

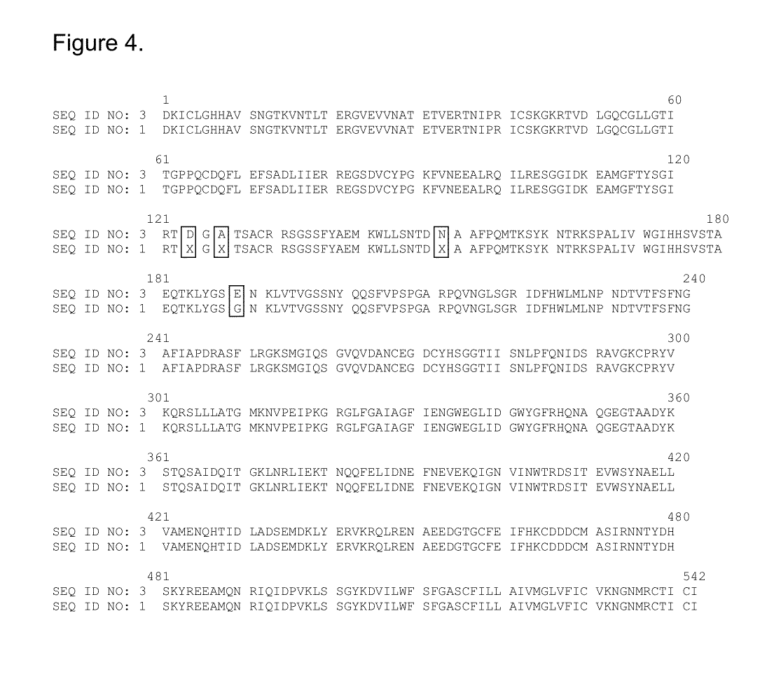

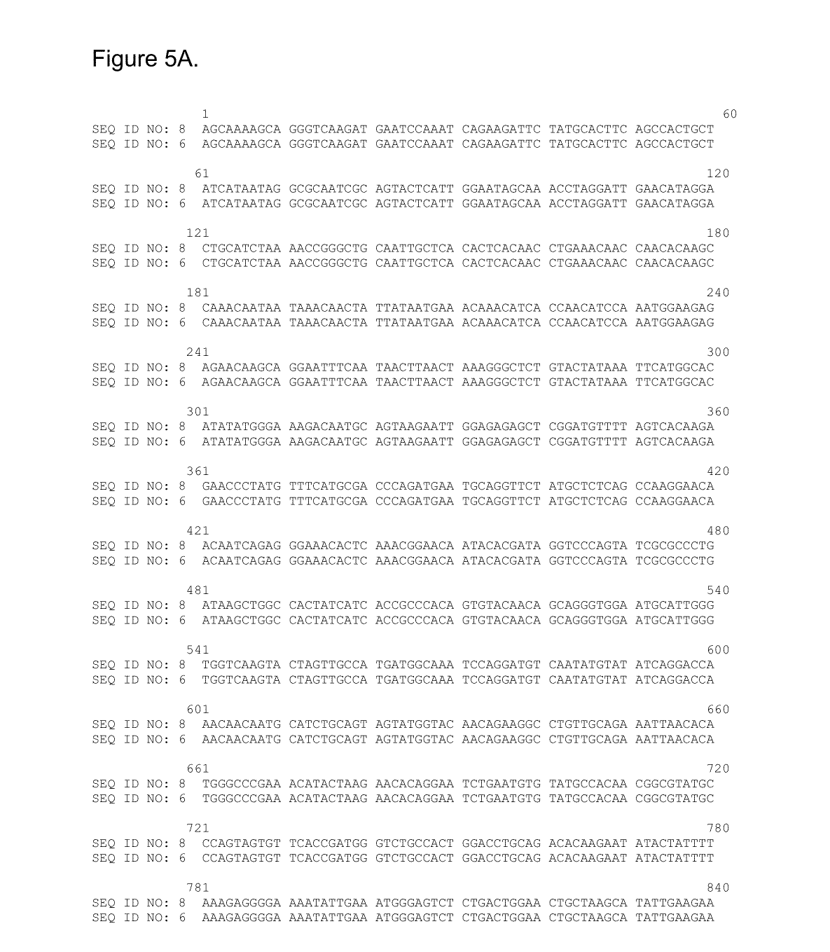

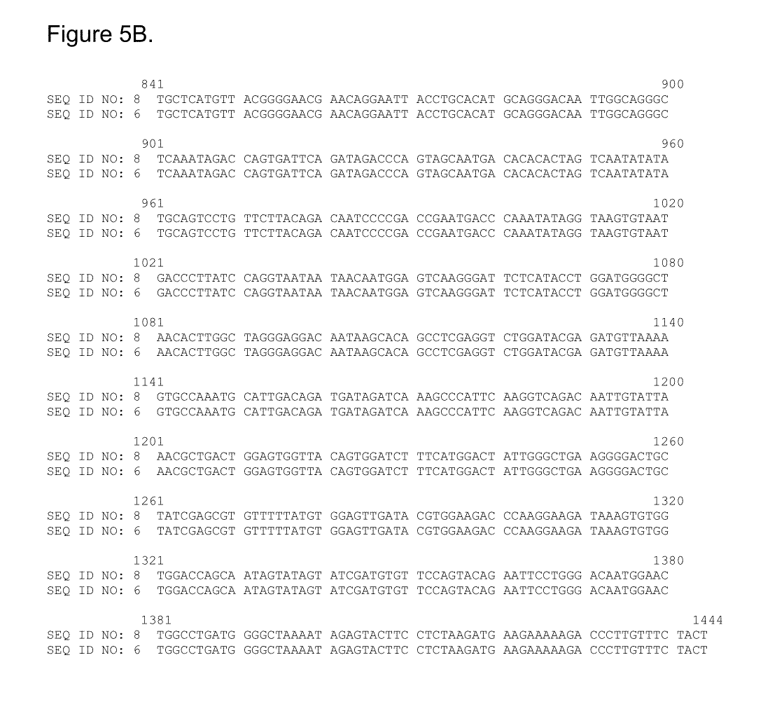

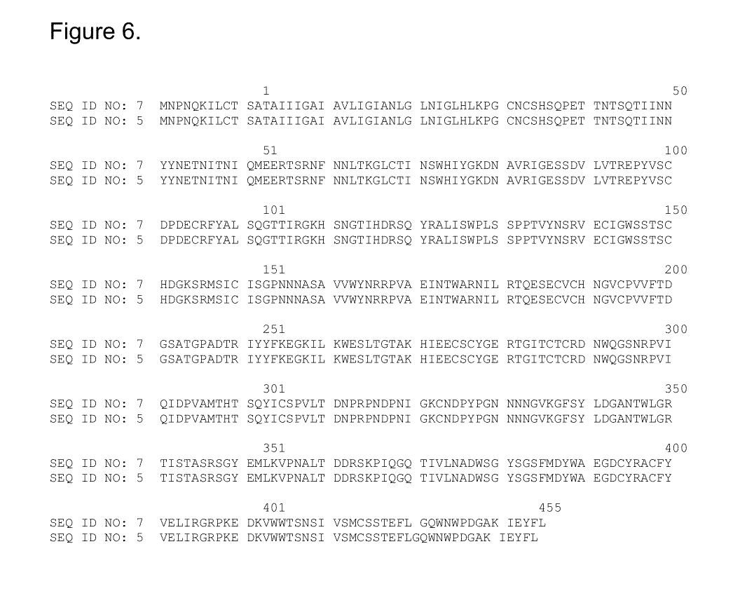

The polynucleotide encoding the Hemagglutinin (HA) and neuraminidase (NA) of viral RNAs isolated from egg-adapted A/Anhui/1/2013 V7 were sequenced. Comparison of the sequences from the A/Anhui/1/2013 V7 with those published for the wt H7N9 from human isolate revealed that the HA genes contained egg adaptation sequence changes. FIG. 3A and FIG. 3B depicts an alignment of the nucleotide sequence of the polynucleotide encoding HA in wt A/Anhui/1/2013 with the nucleotide sequence of the polynucleotide encoding HA in variant V7. Nucleotides 442, 448, 520, and 641 are boxed to highlight that these are the nucleotides that are changed when compared to the reference sequence. FIG. 3A shows nucleotides 1 to 900 and FIG. 3B shows nucleotides 901 to 1733. FIG. 4 depicts an alignment of the amino acid sequence of the HA polypeptide in wt A/Anhui/1/2013 with the amino acid sequence obtained by translating the nucleotide sequence of variant V7. In FIG. 4, X123=N or D; X125=A or T; X149=N or D. As seen in FIG. 4, when the polynucleotides were translated, it was determined that the HA polynucleotide of variant V7 had mutations that correspond to positions 123, 125 and 149 (H7 numbering) of the HA polypeptide. In FIG. 4 the amino acids at these positions are boxed to indicate that these are the amino acids that are modified in variant V7 when compared to wt A/Anhui/1/2013. As shown in FIG. 5A and FIG. 5B, alignment of the sequences obtained for the NA polynucleotide revealed that the NA in variant V7 had the same sequence as the wt virus. FIG. 6 presents an alignment of the amino acid sequence of the NA polypeptide from wt A/Anhui/1/2013 with the amino acid sequence of the NA polypeptide in variant V7.

### Example 4

### The Selected Vaccine Candidate is Attenuated but Immunogenic in Ferrets

To evaluate the attenuation phenotype of ca A/Anhui/1/2013 variants, ferrets were inoculated with 7.0 log₁₀FFU of V1 and V7 intranasally in 0.2 ml of dose volume and virus replication in the upper and lower respiratory tracts of ferrets was determined by EID₅₀ assay. 50 percent Embryo Infectious Dose or EID50 provides a unit of measurement of infectivity. One EID50 unit is the amount of virus that will infect 50 percent of inoculated eggs. As shown in Table 4, below, both V1 and V7 variants replicated in the nasal turbinates (NT) tissues with an average titer of 3.8 and 4.4 log₁₀ EID₅₀/ml respectively, but no virus was detected in the lungs.

TABLE 4
Replication and Immunogenicity of ca A/CA/7/09 (V7) in Ferrets

| | | Virus titer | | GMT HAI titer of | |
|---|---|---|---|---|---|
| | | (log₁₀EID₅₀/ | | ferret serum against | |
| | | g ± SE) | | A/Anhui/1/2013 | |
| 6:2 variant | HA Sequence | NT | Lung | V1 | V7 |
| V1 | wt | 3.8 ± 0.58 | <1.5 | 64 | 40 |
| V7 | N123D/G189E | 4.4 ± 0.14 | <1.5 | 128 | 81 |

These data confirmed that the ca A/Anhui/1/2013 variants were attenuated in ferrets, a characteristic phenotype conferred by the six internal protein gene segments of MDV-A.

To evaluate the immunogenicity and antigenicity of the V7 variant, ferrets were inoculated with V7 intranasally as described above. The post-infection serum was collected on day 14 and antibody titers were evaluated by HAI assay (Table 4). V7 induced a good HAI antibody titer of 81 to the homologous virus, and cross-reacted well to the reference V1 virus. Accordingly, ferret antiserum against wt A/Anhui/1/2013 V1 also cross-reacted well to the V7 variant. These data demonstrate that the N123D/G189E substitution (V7) in the HA of ca A/Anhui/1/2013 conferred high growth in eggs without altering virus antigenicity or immunogenicity, making it a suitable vaccine virus for the novel H7N9 virus.

Based on the data obtained from various H7N9 ca A/Anhui/1/2013 vaccine variants, the V7 with the N123D and G189E changes in the HA has been selected as the vaccine strain for manufacture. The nucleotide sequence of the polynucleotide encoding HA of the V7 variant is set forth in SEQ ID NO: 2, and the amino acid sequence of the HA polypeptide is set forth in SEQ ID NO: 4. The V7 variant has yield of ˜8.7 log₁₀FFU/ml in eggs, immunogenic in seronegative ferrets and has the correct antigenicity.

### Example 5

### Comparison of HA Protein Yield of A/Anhui/1/2011 Variants with the Current Inactivated H7N9 Vaccine Candidate RG32A

A PR8 reassortant (A/Shanghai/2/2013, RG32A), containing the 6 internal gene segments from A/Puerto Rico/8/34 (PR8), the HA and NA gene segments from A/Shanghai/2/2013 (H7N9) whose HA amino acid sequence is identical to A/Anhui/1/2013, was generated by CDC for manufacturing the inactivated H7N9 vaccines. It was expected that the HA protein yield of the H7N9 reassortants would be greatly improved by the V7 amino acid substitutions in the HA. To evaluate this, 6:2 reassortant influenza viruses comprising the 6 internal genome segments from PR8, the NA gene segment from A/Anhui/1/2013, and the HA gene segments from A/Anhui/1/2013-V1 or V7 were generated by plasmid rescue. These 6:2 PR8 V1 and V7 variants, along with RG32A, 6:2 MDVA-V1 and V7, were expanded in embryonated chicken eggs and their titers were determined by FFA as shown in Table 5. PR8-V7 showed significantly higher titer (yield) in eggs than PR8-V1 and RG32A.

TABLE 5
Titer of 6:2 Reassortant H7N9 Passaged in Embryonated Chicken Eggs

| | Titer in eggs |
|---|---|
| Virus | (log₁₀ FFU/ml) |
| 6:2 PR8-A/Anhui/1/2013(V1) | 8.1* |
| 6:2 PR8-A/Anhui/1/2013(V7) | 8.9 |
| A/shanghai/2/2013 (RG32A) | 7.9* |
| 6:2 MDVA-A/Anhui/1/2013(V1) | 7.2 |
| 6:2 MDVA-A/Anhui/1/2013(V7) | 8.6 |

*The viruses exhibited both small and big plaques, indicating that egg adaptation have caused sequence variations and may have improved the titers to some extent.

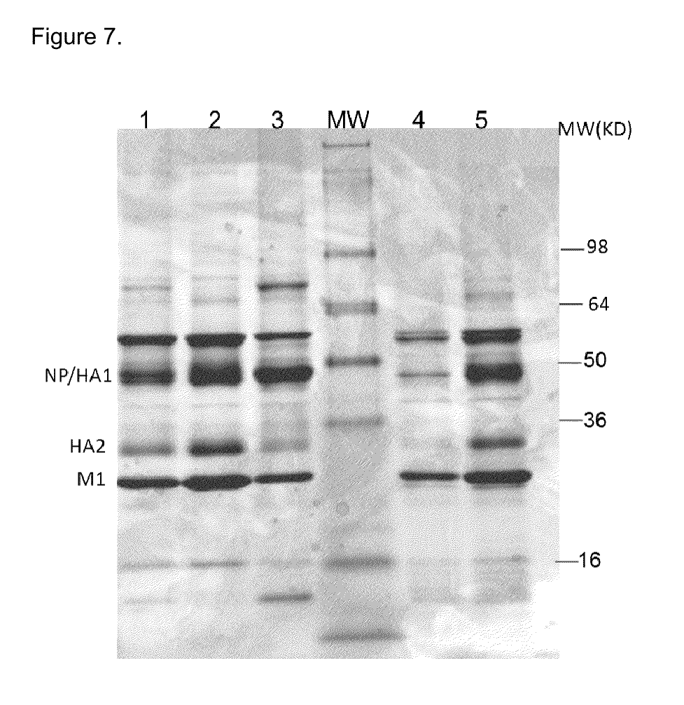

Allantoic fluid (30 ml) harvested from infected eggs was pelleted through 0.3 ml 25% sucrose cushion at 25 k rpm for 2 hrs. The viral pellet was resuspended in PBS (0.3 ml). An equivalent amount of each of the viral suspensions was loaded on 4-20% polyacrylamide gel and stained with Coomassie blue. FIG. 7 depicts an image of a stained gel comprising the viral polypeptides. Lane 1:6:2 PR8-A/Anhui/1/2013(V1); Lane 2: 6:2 PR8-A/Anhui/1/2013(V7); Lane 3: A/shanghai/2/2013 (RG32A); Lane 4: 6:2 MDVA-A/Anhui/1/2013(V1); Lane 5: 6:2 MDVA-A/Anhui/1/2013(V7). As shown in FIG. 7, corresponding to the virus titers in eggs, the PR-V7 had apparently higher HA protein and other viral proteins than the PR8-V1 and RG32A reassortants. Similarly, the LAIV vaccine candidate 6:2 MDVA-V7 had higher vial protein yield than the corresponding V1. The results demonstrated that the two amino acid substitutions in the polynucleotide encoding the HA in the V7 variant (N123D/G189E) greatly improved the viral growth and HA protein yield from embryonated chicken eggs, suggesting the potential of using the V7 variant in the manufacturing of inactivated H7N9 vaccines.

In summary, based on the data obtained from various H7N9 A/Anhui/1/2013 vaccine variants, the V7 with the N123D and G189E changes in the HA has been selected as the vaccine strain for manufacture.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A modified hemagglutinin (HA) polynucleotide encoding a modified HA polypeptide comprising one or more of:
N or D at position 123;
A or T at position 125;
N or D at position 149;
A or T at position 151;
K or N at position 184;
N or E at position 189;
N or D at position 190;
R or S at position 211; or
N or D at position 215
of an HA polypeptide amino acid sequence from a wild-type A/Anhui/1/2013 virus (set forth in SEQ ID NO: 1).

2. The modified HA polynucleotide of claim 1, wherein the modified HA polypeptide comprises one or more of D at position 123, T at position 125, D at position 149, T at position 151, N at position 184, E at position 189, D at position 190, S at position 211, and D at position 215.

3. The modified HA polynucleotide of claim 2, wherein the modified HA polypeptide comprises D at position 123, A at position 125, N at position 149, and E at position 189.

4. A modified HA polynucleotide encoding the polypeptide set forth in SEQ ID NO: 3.

5. A modified HA polypeptide derived from a wild-type A/Anhui/1/2013 virus, the modified HA polypeptide comprising one or more of:
N or D at position 123;
A or T at position 125;
N or D at position 149;
A or T at position 151;
K or N at position 184;
G or E at position 189;
N or D at position 190;
R or S at position 211; or
N or D at position 215.
of an HA polypeptide amino acid sequence from a wild-type A/Anhui/1/2013 virus (set forth in SEQ ID NO: 1).

6. The modified HA polypeptide of claim 5, wherein the modified HA polypeptide comprises one or more of D at position 123, T at position 125, D at position 149, T at position 151, N at position 184, E at position 189, D at position 190, S at position 211, and D at position 215.

7. The modified HA polypeptide of claim 6, wherein the modified HA polypeptide comprises D at position 123, A at position 125, N at position 149, and E at position 189.

8. The modified HA polypeptide of claim 7, wherein the polypeptide comprises E at position 189.

9. A vector comprising the modified HA polynucleotide of claim 1.

10. A cell comprising the vector of claim 9.

11. A reassortant recombinant virus comprising the modified HA polynucleotide of claim 1.

12. A reassortant recombinant virus comprising a modified polynucleotide encoding the modified HA polypeptide of claim 5.

13. A 6:2 reassortant recombinant virus comprising a polynucleotide encoding the modified HA polypeptide of claim 5.

14. An isolated virus-like particle comprising a polynucleotide encoding the modified HA polypeptide of claim 5.

15. An immunogenic composition comprising a polynucleotide encoding the modified HA polypeptide of claim 5.

16. An immunogenic composition comprising the reassortant recombinant virus of claim 11.

17. A method for inducing an immune response against an H7N9 virus, the method comprising administering to a subject the immunogenic composition of claim 15.

18. A kit comprising the immunogenic composition of claim 15.

19. The modified HA polynucleotide of claim 1, wherein a virus comprising the modified polynucleotide has enhanced growth in eggs relative to a reference.

20. A method of preventing or treating an H7N9 viral infection, the method comprising administering to a subject having or at risk of acquiring an H7N9 viral infection an effective amount of the immunogenic composition of claim 15.


SEQUENCE LISTING (8 sequences)

SEQ ID NO: 1 - protein, 542 aa, Influenza A virus
  MOD_RES (123)..(123): Asn or Asp
DKICLGHHAVSNGTKVNTLTERGVEVVNATETVERTNIPRICSKGKRTVDLGQCGLLGTITGPPQCDQFLEFSADLIIERREGSDVCYPGKFVNEEALRQILRESGGIDKEAMGFTYSGIRTXGXTSACRRSGSSFYAEMKWLLSNTDXAAFPQMTKSYKNTRKSPALIVWGIHHSVSTAEQTKLYGSGNKLVTVGSSNYQQSFVPSPGARPQVNGLSGRIDFHWLMLNPNDTVTFSFNGAFIAPDRASFLRGKSMGIQSGVQVDANCEGDCYHSGGTIISNLPFQNIDSRAVGKCPRYVKQRSLLLATGMKNVPEIPKGRGLFGAIAGFIENGWEGLIDGWYGFRHQNAQGEGTAADYKSTQSAIDQITGKLNRLIEKTNQQFELIDNEFNEVEKQIGNVINWTRDSITEVWSYNAELLVAMENQHTIDLADSEMDKLYERVKRQLRENAEEDGTGCFEIFHKCDDDCMASIRNNTYDHSKYREEAMQNRIQIDPVKLSSGYKDVILWFSFGASCFILLAIVMGLVFICVKNGNMRCTICI

SEQ ID NO: 2 - DNA, 1733 bp, Influenza A virus
agcaaaagcaggggatacaaaatgaacactcaaatcctggtattcgctcagattgcgatcattccaacaaatgcagacaaaatctgcctcggacatcatgccgtgtcaaacggaaccaaagtaaacacattaactgaaagaggagtggaagtcgtcaatgcaactgaaacagtggaacgaacaaacatccccaggatctgctcaaaagggaaaaggacagttgacctcggtcaatgtggactcctggggacaatcactggaccacctcaatgtgaccaattcctagaattttcagccgatttaattattgagaggcgagaaggaagtgatgtctgttatcctgggaaattcgtgaatgaagaagctctgaggcaaattctcagagaatcaggcggaattgacaaggaagcaatgggattcacatacagtggaataagaactratggarcaaccagtgcatgtaggagatcaggatcttcattctatgcagaaatgaaatggctcctgtcaaacacagatratgctgcattcccgcagatgactaagtcatataaaaatacaagaaaaagcccagctctaatagtatgggggatccatcattccgtatcaactgcagagcaaaccaagctatatgggagtggaaacaaactggtgacagttgggagttctaattatcaacaatcttttgtaccgagtccaggagcgagaccacaagttaatggtctatctggaagaattgactttcattggctaatgctaaatcccaatgatacagtcactttcagtttcaatggggctttcatagctccagaccgtgcaagcttcctgagaggaaaatctatgggaatccagagtggagtacaggttgatgccaattgtgaaggggactgctatcatagtggagggacaataataagtaacttgccatttcagaacatagatagcagggcagttggaaaatgtccgagatatgttaagcaaaggagtctgctgctagcaacagggatgaagaatgttcctgagattccaaagggaagaggcctatttggtgctatagcgggtttcattgaaaatggatgggaaggcctaattgatggttggtatggtttcagacaccagaatgcacagggagagggaactgctgcagattacaaaagcactcaatcggcaattgatcaaataacaggaaaattaaaccggcttatagaaaaaaccaaccaacaatttgagttgatagacaatgaattcaatgaggtagagaagcaaatcggtaatgtgataaattggaccagagattctataacagaagtgtggtcatacaatgctgaactcttggtagcaatggagaaccagcatacaattgatctggctgattcagaaatggacaaactgtacgaacgagtgaaaagacagctgagagagaatgctgaagaagatggcactggttgctttgaaatatttcacaagtgtgatgatgactgtatggccagtattagaaataacacctatgatcacagcaaatacagggaagaggcaatgcaaaatagaatacagattgacccagtcaaactaagcagcggctacaaagatgtgatactttggtttagcttcggggcatcatgtttcatacttctagccattgtaatgggccttgtcttcatatgtgtaaagaatggaaacatgcggtgcactatttgtatataagtttggaaaaaaacacccttgtttctact

SEQ ID NO: 3 - protein, 542 aa, Influenza A virus
DKICLGHHAVSNGTKVNTLTERGVEVVNATETVERTNIPRICSKGKRTVDLGQCGLLGTITGPPQCDQFLEFSADLIIERREGSDVCYPGKFVNEEALRQILRESGGIDKEAMGFTYSGIRTDGATSACRRSGSSFYAEMKWLLSNTDNAAFPQMTKSYKNTRKSPALIVWGIHHSVSTAEQTKLYGSENKLVTVGSSNYQQSFVPSPGARPQVNGLSGRIDFHWLMLNPNDTVTFSFNGAFIAPDRASFLRGKSMGIQSGVQVDANCEGDCYHSGGTIISNLPFQNIDSRAVGKCPRYVKQRSLLLATGMKNVPEIPKGRGLFGAIAGFIENGWEGLIDGWYGFRHQNAQGEGTAADYKSTQSAIDQITGKLNRLIEKTNQQFELIDNEFNEVEKQIGNVINWTRDSITEVWSYNAELLVAMENQHTIDLADSEMDKLYERVKRQLRENAEEDGTGCFEIFHKCDDDCMASIRNNTYDHSKYREEAMQNRIQIDPVKLSSGYKDVILWFSFGASCFILLAIVMGLVFICVKNGNMRCTICI

SEQ ID NO: 4 - DNA, 1733 bp, Influenza A virus
agcaaaagcaggggatacaaaatgaacactcaaatcctggtattcgctctgattgcgatcattccaacaaatgcagacaaaatctgcctcggacatcatgccgtgtcaaacggaaccaaagtaaacacattaactgaaagaggagtggaagtcgtcaatgcaactgaaacagtggaacgaacaaacatccccaggatctgctcaaaagggaaaaggacagttgacctcggtcaatgtggactcctggggacaatcactggaccacctcaatgtgaccaattcctagaattttcagccgatttaattattgagaggcgagaaggaagtgatgtctgttatcctgggaaattcgtgaatgaagaagctctgaggcaaattctcagagaatcaggcggaattgacaaggaagcaatgggattcacatacagtggaataagaactgatggagcaaccagtgcatgtaggagatcaggatcttcattctatgcagaaatgaaatggctcctgtcaaacacagataatgctgcattcccgcagatgactaagtcatataaaaatacaagaaaaagcccagctctaatagtatgggggatccatcattccgtatcaactgcagagcaaaccaagctatatgggagtgaaaacaaactggtgacagttgggagttctaattatcaacaatcttttgtaccgagtccaggagcgagaccacaagttaatggtctatctggaagaattgactttcattggctaatgctaaatcccaatgatacagtcactttcagtttcaatggggctttcatagctccagaccgtgcaagcttcctgagaggaaaatctatgggaatccagagtggagtacaggttgatgccaattgtgaaggggactgctatcatagtggagggacaataataagtaacttgccatttcagaacatagatagcagggcagttggaaaatgtccgagatatgttaagcaaaggagtctgctgctagcaacagggatgaagaatgttcctgagattccaaagggaagaggcctatttggtgctatagcgggtttcattgaaaatggatgggaaggcctaattgatggttggtatggtttcagacaccagaatgcacagggagagggaactgctgcagattacaaaagcactcaatcggcaattgatcaaataacaggaaaattaaaccggcttatagaaaaaaccaaccaacaatttgagttgatagacaatgaattcaatgaggtagagaagcaaatcggtaatgtgataaattggaccagagattctataacagaagtgtggtcatacaatgctgaactcttggtagcaatggagaaccagcatacaattgatctggctgattcagaaatggacaaactgtacgaacgagtgaaaagacagctgagagagaatgctgaagaagatggcactggttgctttgaaatatttcacaagtgtgatgatgactgtatggccagtattagaaataacacctatgatcacagcaaatacagggaagaggcaatgcaaaatagaatacagattgacccagtcaaactaagcagcggctacaaagatgtgatactttggtttagcttcggggcatcatgtttcatacttctagccattgtaatgggccttgtcttcatatgtgtaaagaatggaaacatgcggtgcactatttgtatataagtttggaaaaaaacacccttgtttctact

SEQ ID NO: 5 - protein, 465 aa, Influenza A virus
MNPNQKILCTSATAIIIGAIAVLIGIANLGLNIGLHLKPGCNCSHSQPETTNTSQTIINNYYNETNITNIQMEERTSRNFNNLTKGLCTINSWHIYGKDNAVRIGESSDVLVTREPYVSCDPDECRFYALSQGTTIRGKHSNGTIHDRSQYRALISWPLSSPPTVYNSRVECIGWSSTSCHDGKSRMSICISGPNNNASAVVWYNRRPVAEINTWARNILRTQESECVCHNGVCPVVFTDGSATGPADTRIYYFKEGKILKWESLTGTAKHIEECSCYGERTGITCTCRDNWQGSNRPVIQIDPVAMTHTSQYICSPVLTDNPRPNDPNIGKCNDPYPGNNNNGVKGFSYLDGANTWLGRTISTASRSGYEMLKVPNALTDDRSKPIQGQTIVLNADWSGYSGSFMDYWAEGDCYRACFYVELIRGRPKEDKVWWTSNSIVSMCSSTEFLGQWNWPDGAKIEYFL

SEQ ID NO: 6 - DNA, 1444 bp, Influenza A virus
agcaaaagcagggtcaagatgaatccaaatcagaagattctatgcacttcagccactgctatcataataggcgcaatcgcagtactcattggaatagcaaacctaggattgaacataggactgcatctaaaaccgggctgcaattgctcacactcacaacctgaaacaaccaacacaagccaaacaataataaacaactattataatgaaacaaacatcaccaacatccaaatggaagagagaacaagcaggaatttcaataacttaactaaagggctctgtactataaattcatggcacatatatgggaaagacaatgcagtaagaattggagagagctcggatgttttagtcacaagagaaccctatgtttcatgcgacccagatgaatgcaggttctatgctctcagccaaggaacaacaatcagagggaaacactcaaacggaacaatacacgataggtcccagtatcgcgccctgataagctggccactatcatcaccgcccacagtgtacaacagcagggtggaatgcattgggtggtcaagtactagttgccatgatggcaaatccaggatgtcaatatgtatatcaggaccaaacaacaatgcatctgcagtagtatggtacaacagaaggcctgttgcagaaattaacacatgggcccgaaacatactaagaacacaggaatctgaatgtgtatgccacaacggcgtatgcccagtagtgttcaccgatgggtctgccactggacctgcagacacaagaatatactattttaaagaggggaaaatattgaaatgggagtctctgactggaactgctaagcatattgaagaatgctcatgttacggggaacgaacaggaattacctgcacatgcagggacaattggcagggctcaaatagaccagtgattcagatagacccagtagcaatgacacacactagtcaatatatatgcagtcctgttcttacagacaatccccgaccgaatgacccaaatataggtaagtgtaatgacccttatccaggtaataataacaatggagtcaagggattctcatacctggatggggctaacacttggctagggaggacaataagcacagcctcgaggtctggatacgagatgttaaaagtgccaaatgcattgacagatgatagatcaaagcccattcaaggtcagacaattgtattaaacgctgactggagtggttacagtggatctttcatggactattgggctgaaggggactgctatcgagcgtgtttttatgtggagttgatacgtggaagacccaaggaagataaagtgtggtggaccagcaatagtatagtatcgatgtgttccagtacagaattcctgggacaatggaactggcctgatggggctaaaatagagtacttcctctaagatgaagaaaaagacccttgtttctact

SEQ ID NO: 7 - protein, 465 aa, Influenza A virus; identical to SEQ ID NO: 5

SEQ ID NO: 8 - DNA, 1444 bp, Influenza A virus; identical to SEQ ID NO: 6